(19) United States
(12) Patent Application Publication
(10) Pub. No.: US 2008/0170996 A1
(43) Pub. Date: Jul. 17, 2008

(54) Compositions and Methods for Stimulation of Lung Innate Immunity

(75) Inventors: Burton F. Dickey, Houston, TX (US); Michael Tuvim, Houston, TX (US); Cecilia G. Clement, The Woodlands, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of the University of Texas System, Austin, TX (US)

(21) Appl. No.: 11/830,622

(22) Filed: Jul. 30, 2007

Related U.S. Application Data
(60) Provisional application No. 60/833,857, filed on Jul. 28, 2006.

(51) Int. Cl. A61K 9/12; A61K 35/66; A61P 31/00

(52) U.S. Cl. 424/45; 424/780

(57) ABSTRACT

Embodiments of the invention include compositions, formulations and methods for the enhancement of a subject's biological defenses against infection, for example the subject's innate immunity against infection. Aspects of the invention provide a rapid and temporal enhancement or augmentation of biological defenses against microbial infection.

This application claims priority to U.S. Provisional Patent application Ser. No. 60/833,857 filed Jul. 28, 2006, entitled “COMPOSITIONS AND METHODS FOR STIMULATION OF LUNG INNATE IMMUNITY,” which is incorporated herein by reference in its entirety.

This invention was made with government support under HL072984, CA105352, and CA016672 awarded by the National Institutes of Health. The government has certain rights in the invention.

### BACKGROUND OF THE INVENTION

I. Field of the Invention

The present invention relates to the general fields of microbiology, immunology, physiology, and medicine. More particularly the compositions and methods of the invention relate to modulation of innate immunity in the lungs of a subject for the treatment or attenuation of microbial infection or invasion.

II. Background

The lungs are a common site of serious infections, both in healthy subjects and in those who are immunocompromised. In addition, the lungs are a likely portal of entry for bioterror agents. The susceptibility of the lungs to infection derives from the architectural requirements of gas exchange, resulting in continuous exposure of a large surface area to the outside environment while imposing a minimal barrier to gas diffusion. The demands of gas exchange preclude protective strategies such as encasement of the surface in an impermeable barrier, as in the skin, or continuous generation of a heavy blanket of mucus, as in the gastrointestinal tract. Furthermore, infiltration of lung tissue with inflammatory cells and edema fluid to fight infection results in serious impairment of gas exchange, so recruitment of cells and proteins from the circulation can be viewed as a defensive strategy of last resort.

Despite their structural vulnerability, the lungs generally defend themselves successfully against infection through a variety of mechanical, humoral, and cellular mechanisms. Lung defenses can be divide into at least two structural features (1) conducting airways and (2) gas-exchanging alveoli. At baseline, the lungs are protected against infection by (a) impaction and sedimentation of inhaled pathogens in the airways, followed by clearance through cough, sneezing and mucociliary action; (b) the antimicrobial effects of antibodies and innate immune polypeptides secreted into the lung lining fluid; and (c) the phagocytic activity of alveolar macrophages, which account for more than 95% of the leukocytes in human and pathogen-free mouse lungs (Martin and Frevert, 2005). In the presence of inflammatory stimuli, the lungs are capable of rapidly recruiting neutrophils and lymphocytes from the circulation, and the airway epithelium undergoes remarkable structural changes termed “metaplasia.” It is thought that the structural changes of metaplasia are accompanied by changes in antimicrobial defensive function, and some evidence exists to support this hypothesis (Martin and Frevert, 2005). Within hours of exposure to the Th2 cytokine IL-13 or antigens to which the immune system has been sensitized, secretory cells of the airway increase their height in association with filling of their cytoplasm with glycoconjugates, which are visible by light microscopy after histochemical staining, and with large electron lucent secretory granules, which are visible by electron microscopy. Many of these structural changes can be ascribed to the increased synthesis of the gel-forming mucin Muc5ac, but additional molecular changes such as increased synthesis of Gob-5, acidic mammalian chitinase, and the A3 adenosine receptor indicate a broader phenotypic response. The presumed adaptive value of this structural and molecular plasticity of the airway epithelium is augmented defense against microbial pathogens. This inference is supported by the roles in inflammatory metaplasia of molecules such as complement and toll like receptors that have primary roles in microbial defense. However, the functional significance of these structural and molecular changes in defense against pathogens is mostly unknown.

Excessive mucin secretion can lead to blockage of small airways, which is a serious problem in common obstructive disease such as asthma, cystic fibrosis, and COPD. In contrast to the airways, alveolar epithelial cells do not undergo substantial structural change during inflammation. In fact, the alveolar environment is relatively anti-inflammatory, thereby preventing an influx of cells and fluid that would interfere with gas exchange. Surfactant lipids and proteins have anti-inflammatory activities, and alveolar macrophages are capable of ingesting particles and pathogens without triggering major inflammatory responses (Martin and Frevert, 2005).

One example of these protective mechanisms can be seen in the lungs response to viral infections, which can be applied to other microbes such as bacteria and fungi. Respiratory viral infections are very common and involve the nose and conducting airways. In normal hosts these upper respiratory viral infections generally cause little morbidity and mortality, although some viruses such as RSV cause prolonged symptoms and promote the asthma phenotype. Occasionally, endemic respiratory viruses may infect the lower respiratory tract (alveoli) in a more serious syndrome called pneumonia. The mechanisms that generally limit viral infections to the upper respiratory tract probably involve physical barriers to the penetration of infectious aerosols, the activity of antimicrobial polypeptides in airway lining fluid, and cellular mechanisms. Small numbers of persons die in the United States each year from endemic respiratory viruses, and many more from annual epidemic influenza.

For reasons that are not yet fully understood, influenza viruses sometimes evolve to become highly pathogenic, killing large numbers of normal hosts. Effective adaptive immune vaccines are generally available in the developed world against influenza, though there is increasing concern about the possibility of the sudden emergence of a highly transmissible and highly pathogenic influenza virus due to mixing of viral strains in farm animals in Asia, coupled with frequent worldwide travel of humans. Other viruses, such as RSV, have been resistant to the development of effective vaccines. In addition to well-recognized endemic and epidemic viruses, emerging viral infections have been important causes of pneumonia. For example, a hantavirus pneumonia syndrome was recognized in the American Southwest in 1993 with a case-fatality rate of 37%. In 2003, the SARS virus apparently jumped from bats to civets to humans in China, causing more than 8,000 cases of pneumonia worldwide with a case-fatality rate of 10%. Based on these occurrences, it is reasonable to expect that additional emergent respiratory viral infections will be identified in the future. In addition, both hantavirus and SARS virus are classified as Category C bioweapon agents.

There is a need for additional compositions and methods for treating, inhibiting, attenuating, or preventing infection of a subject via the respiratory route. This application describes various compositions and methods for the protection from and treatment before and after infection by an inhaled microbe, be it viral, bacterial, fungal, etc. In certain aspects the compositions, formulations, and methods do not require an adaptive or acquired immune response to be effective, and therefore can be used against a broad spectrum of pathogenic or potentially pathogenic organisms.

### SUMMARY OF THE INVENTION

Embodiments of the invention include compositions, formulations and methods for the enhancement of a human subject's biological defenses against infection, for example the subject's innate immunity against infection. Aspects of the invention provide a rapid and temporal enhancement or augmentation of biological defenses against microbial infection. The enhancement of the innate immunity of a subject attenuates microbial infections. Attenuation can be by inhibiting, treating, or preventing infection. Aspects of the invention enhance the innate immune defenses of the lung and respiratory tract of a subject. In certain aspects the subject is administered a microbial lysate that enhances the subject's biological defenses. In a further embodiment of the invention the microbial lysate is produced from a non-pathogenic microbe. A non-pathogenic microbe is a microbe that typically does not cause disease in a subject exposed to the microbe, particularly via infecting the lungs. Disease is defined as the significant impairment in the function of a tissue, an organ, or a system of a subject. A microbe need not be a microbe of the same kind, genus or species from which protection or therapy is sought. In certain embodiments the microbial lysate is comprised of a heterologous or second microbe, i.e., a microbe that differs from a first microbe or class of microbes from which protection or treatment is sought, e.g., non-pathogenic microbial lysate (e.g., NTHi lysate) as compared to a potentially infecting pathogenic microbe(s) (B. anthracis, influenza, Aspergillus fumigatus, etc.). In certain aspects, a lysate may comprise a mixture of microbial lysates or a mixture of fractions of two or more microbial lysates. In still further embodiments, the microbial lysate can be of a second pathogenic microbe or a second non-pathogenic microbe, or a mixture of both.

Embodiments of the invention include methods of attenuating respiratory infection by a pathogenic first microbe in a human subject who has or is at risk for developing such an infection, the method comprising administering a non-pathogenic microbial lysate, wherein said lysate is prepared from an essentially non-pathogenic second microbe, to the subject by aerosol inhalation in an amount sufficient to induce innate immunity in the subject to said first microbe and thereby attenuate the respiratory infection. Aspects of the invention include the enhancement of innate immunity within the lungs. In a further aspect the microbial lysate is administered by inhalation. The present invention includes the preparation and use of a microbial extract or lysate that contains multiple molecular components that stimulate or cause the augmentation of various biological pathways in a subject, particularly those of the respiratory system. A further aspect of the invention includes inducing protection from microbial infection with minimal toxicity to a subject.

In certain aspects, the pathogenic first microbe is a virus, a bacteria, or a fungus. In another aspect the pathogenic microbe is a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses. In still a further embodiment, the virus includes, but is not limited to Parainfluenza, Influenza, H5N1, Marburg, Ebola, Severe acute respiratory syndrome coronavirus, Yellow fever virus, Human respiratory syncytial virus, Hantavirus, or Vaccinia virus.

In yet a further aspect, the pathogenic microbe is a bacteria. A bacteria can be an intracellular, a gram positive or a gram negative bacteria. In a further aspect, the bacteria includes, but is not limited to a Staphylococcus, a Bacillus, a Francisella, or a Yersinia bacteria. In still a further aspect, the bacteria is Bacillus anthracis, Yersinia pestis, Francisella tularensis, or Staphylococcus aureas. In certain embodiments, a bacteria is Bacillus anthracis and/or Staphylococcus aureas. In still a further aspect, a bacteria is a drug resistant bacteria, such as a multiple drug resistant Staphylococcus aureas.

In still anther aspect, the pathogenic first microbe is a fungus. The fungus can include, but is not limited to members of the family Aspergillus, Candida, Crytpococus, Histoplasma, Coccidioides, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales. The family Aspergillus includes, but is not limited to Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, Aspergillus versicolor and the like. The family Candida includes, but is not limited to C. albicans, C. dubliniensis, C. glabrata, C. guilliermondii, C. kefyr, C. krusei, C. lusitaniae, C. milleri, C. oleophila, C. parapsilosis, C. tropicalis, C. utilis, and the like.

Embodiments of the invention also include pharmaceutically acceptable compositions comprising a lysate of an essentially non-pathogenic microbe, an anti-inflammatory agent and one or more pharmaceutical excipients, wherein said composition is sterile and essentially free of pathogenic microbes. A microbial lysate is typically sonicated; homogenized; irradiated; lysed by barometric, pneumatic, detergents, or enzymatic methods and combinations thereof. In a particular aspect the microbial lysate is UV irradiated before, during, or after lysis. The microbial lysate can include, but is not limited to a bacterial, fungal, or viral lysate. In certain embodiments the microbial lysate is a bacterial lysate. The microorganism from which the lysate is prepared need not be a virulent microorganism, and typically will not be a virulent microorganism. Aspects of the invention include a lysate derived from bacteria having a limited effect on the health of a subject. Limited effect refers to producing minimal adverse reactions and insubstantial impairment in the function of a tissue, an organ, or a system of a subject over a period of at least, at most, or about 1, 2, 3, 4, 5, 6, 7, 8, 9, or 10 days.

Compositions of the invention need not be derived directly from a virulent organism from which protection or therapy is sought. The bacteria can be from the genus Haemophilus, but is not limited to Haemophilus. Bacteria that pose a minimal threat of adverse effects in a subject can be identified. In certain aspects the bacteria is Haemophilus influenzae, particularly non-typeable Haemophilus influenzae (NTHi).

A microbial lysate can have a protein concentration of at least about, about, or at most about 0.5, 1, 1.5, 2, 2.5, 3, 3.5, 4, 4.5, 5, 5.5, 6, 6.5, 7, 7.5, 8, 8.5, 9, 9.5 or 10 mg/ml, including all values and ranges there between. In certain aspects the microbial lysate can have a protein concentration of at least about, about, or at most about 10 mg/ml.

Embodiments of the invention include a microbial lysate that can be administered via the respiratory tract. In certain aspects administration is by inhalation. In a further aspect the composition is aerosolized or in a form that can be inhaled by a subject. In certain embodiments, a lysate composition comprises an anti-inflammatory agent, including steroidal and non-steroidal antiinflammatories (NSAIDs).

Methods of the invention include the (a) augmentation or enhancement of the immune system, e.g., innate immune system, of a subject, and (b) the protection and/or treatment of an individual exposed to a pathogen or organism or microbe, in certain aspects an airborne pathogen or organism or microbe, as well as kits and other compositions that can be used in conjunction with these and related methods. Certain embodiments include methods of enhancing immune responses in the lungs of a subject comprising the steps of (a) obtaining an inhalent comprising a microbial lysate; and (b) administering the microbial lysate to a subject exposed to or at risk of exposure to an airborne organism. The immune response typically comprises production of microbicidal agents, such as, but not limited to reactive oxygen species (ROS), microbiocidal proteins, activation of phagocytic and microbiocidal cells, activation or production of components of the complement system, or combinations thereof. In particular aspects, the methods minimize the induction of mucin secretion or do not stimulate mucin secretion in an amount that is detrimental to the subject. Compositions of the invention can be administered at least, about, or at most 1, 2, 3, 4, 5, 6, 7, 8, 9, 10 or more times. In particular aspect of the invention the compositions are administered after or before a subject is at risk or heightened risk of exposure to a potentially pathogenic or pathogenic organism(s). Such as a soldier on the battlefield or a first responder to an epidemic, pandemic or other emergency situation. Methods of the invention may also include the step of identifying potential exposure of a subject, or identifying a subject exposed to or at risk of exposure to an organism. Identifying risk of exposure can include detecting the presence of a pathogenic or potentially pathogenic organism. Identifying risk of exposure may also include the location of a subject and the assessment of the environment in which the subject is operating, such as, but not limited to a war zone, a region in the midst of a pandemic, or a hospital; particularly in places where microbes, obligate microbes or bioweapons may be present.

Methods of the invention include the administration of a microbial lysate by inhalation or other methods of administration to the upper and/or lower respiratory tract. In certain aspects the microbial lysate is aerosolized or aspirated. The subject can be at risk of exposure to or exposed to an airborne virus, bacteria, or fungus. In certain aspects the pathogenic bacteria is an intracellular, a gram positive or a gram negative bacterium. In certain embodiments the bacteria is a Streptococcus, Staphylococcus, Bacillus, Francisella, or Yersinia. In still further aspects the bacteria is Bacillus anthracis, Yersinia pestis, Francisella tularensis, Streptococcus pnemoniae, Staphylococcus aureas, Pseudomonas aeruginosa, and/or Burkholderia cepacia.

Still further embodiments include methods where the lysate is administered before; after; during; before and after; before, after and during exposure to the organism. The subject can be exposed to a bioweapon or to an opportunistic pathogen. In particular aspects the subject is immunocompromised, such as a cancer patient or an AIDS patient.

Still further embodiments of the invention include a pharmaceutically acceptable aerosol composition prepared by a process comprising the steps of: (a) obtaining a composition of essentially non-pathogenic microbe; (b) treating the composition to kill microbes therein; (c) lysing the microbes to prepare a lysate; and (d) aerosolizing the lysate to prepare the aerosol composition; wherein the aerosol composition is sterile and essentially free of pathogenic microbes. In certain aspects the microbes or microbial lysate is irradiated. In a further aspect, the microbes or microbial lysate is UV irradiated. In still a further aspect, the microbes are lysed by sonication, homogenization, barometric, detergent, enzymatic, or pneumatic methods.

Embodiments of the invention also include methods of preparing a pharmaceutically acceptable aerosol composition comprising the steps of: (a) obtaining a composition of essentially non-pathogenic microbe; (b) treating the composition to kill microbes therein; (c) lysing the microbes to prepare a lysate; and (d) aerosolizing the lysate to prepare the aerosol composition; wherein the aerosol composition is sterile and essentially free of pathogenic microbes. In certain aspects, the microbe is killed by irradiation, such as UV irradiation. In certain aspects the lysate is prepared by sonication, homogenization, barometric, pneumatic, detergent, and/or enzymatic methods.

Other aspects of the invention include the ability to readily produce in large quantities of the inventive compositions.

The terms “attenuating,” “inhibiting,” “reducing,” or “prevention,” or any variation of these terms, when used in the claims and/or the specification includes any measurable decrease or complete inhibition to achieve a desired result, e.g., reduction in post-exposure bacterial load or growth.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Bronchoalveolar lavage (BAL) fluid cell counts from mice treated with NHTi lysate by aerosol.

FIG. 2 Survival After Spn Aerosol Challenge. Groups of six mice each were exposed for one hour to aerosols containing increasing concentrations of Spn, and surviving mice were counted daily.

FIGS. 3A-3D Inflammatory Cell Counts in Bronchoalveolar Lavage Fluid After Spn Challenge or Treatment with NTHi Lysate. Mice were exposed for one hour to Spn aerosols containing 1.0×10⁹ colony forming units (CFU)/ml (A) or 6.1×10¹⁰ CFU/ml (B), for 20 min to the aerosolized NTHi lysate (C) or spn aerosols containing 4×10¹⁰ CFU/ml after exposure to NTHi lysate (D). Groups of five mice each were then sacrificed at the indicated time points, their lungs lavaged with 2 ml of saline solution, total cell counts measured with a hemacytometer, and differential cell counts determined by cytocentrifugation with Wright-Giemsa staining. Shown are the mean ±SEM of the cell counts. No data are available for the high dose Spn challenge after 24 hours because all the mice died (B).

FIG. 4 Survival after Pretreatment with NTHi Lysate Followed by Spn Aerosol Challenge. Mice were pretreated in groups of six with NTHi lysate at various time points or left untreated, then pooled and challenged as a single group with high dose Spn (6.1×10¹⁰ CFU/ml). Survival at seven days as a function of the interval between NTHi treatment and Spn challenge is illustrated.

FIG. 5 Survival after Spn Aerosol Challenge Followed by Post-Challenge Treatment with NTHi Lysate. Mice were challenged as a single group with high dose Spn (3.5×10¹⁰ CFU/ml), then treated in groups of six with NTHi lysate 2 hr or 24 hr after Spn challenge or left untreated.

FIG. 6 Survival after Pretreatment with NTHi Lysate Followed by Intraperitoneal or Intravenous Spn Challenge. Mice were pretreated with NTHi lysate 4 hr before Spn challenge, or left untreated. Groups of six treated and six untreated mice were then each challenged with 10 CFU of Spn injected into the intraperitoneal space (IP) or into the tail vein (IV), and survival at seven days is illustrated.

FIG. 7 Bacterial Counts in the Lungs of Mice Pretreated with NTHi Lysate then Challenged with Spn Aerosol. Mice were pretreated in groups of four with NTHi lysate at various time points or left untreated, then pooled and challenged as a single group with high dose Spn (2.1×10¹⁰ CFU/ml). Lungs were removed immediately after completion of the aerosol challenge, homogenized, and plated for bacterial culture. Shown are the mean ±SEM bacterial counts, with * indicating p<0.05 for treated mice compared to those untreated.

FIGS. 8A-8B Host Survival and Lung Bacterial Counts in Mice Deficient in Alveolar Macrophages and Neutrophils but Treated with NTHi Lysate, then Challenged with Spn Aerosol. Half the mice were given intranasal liposomal clodronate to deplete alveolar macrophages and intravenous rat monoclonal antibodies to deplete neutrophils (M/N). Half of the M/N sufficient mice and half of the M/N deficient mice were treated with NTHi lysate 4 hr before Spn challenge. All of the mice were then pooled and challenged as a single group with high dose Spn (1.5×10¹⁰ CFU/ml). Groups of six mice each were followed for survival at seven days (FIG. 8A), and the lungs of groups of three mice each were removed immediately after completion of the aerosol challenge for bacterial culture (FIG. 8B). Shown for the bacterial counts are the mean ±SEM, with * indicating p<0.05 for M/N sufficient mice treated with NTHi compared to those untreated, and 1 indicating p<0.05 for M/N deficient mice treated with NTHi compared to those untreated.

FIG. 9 Reversed Phase HPLC Analysis of Proteins Present in Bronchoalveolar Lavage Fluid After Treatment with NTHi Lysate. BAL fluid supernatants were collected from the lungs of mice that were untreated (BAL control (ctrl)) or pretreated 48 hr previously with NTHi lysate (BAL day 2), desalted by acetone precipitation, then fractionated on a C-18 column eluted with a 0.1% trifluoroacetic acid and acetonitrile gradient. UV absorbance was monitored at 214 and 295 nm, and representative elution profiles measured at 214 nm are shown in the illustration. Proteins from individual fractions were digested with trypsin, analyzed by LC-MS/MS, and identified by database searching.

FIGS. 10A-10C Identification and Relative Quantification of a Peptide from Chitinase-3-Like Protein Using Isobaric Stable Isotope Tag (iTRAQ) Analysis of Proteins Present in Bronchoalveolar Lavage Fluid After Treatment with NTHi Lysate. BAL fluid supernatants were collected and precipitated as in FIG. 9, then alkylated with methyl methanethiosulfonate, digested with trypsin, and separately derivatized with iTRAQ 114 (BAL control) or iTRAQ 117 (BAL day 2) (Applied Biosystems, Foster City, Calif.). The derivatized digests were then combined and analyzed by nano-LC-MS/MS, and proteins identified by database searching. Shown is a representative total ion chromatogram from 74-96 min displaying the sum of the ion-current at each time point, the mass spectrum at 89.2 min (FIG. 10B), and a high resolution image of the mass spectrum of an ion with a mass/charge ratio of 682.9 (FIG. 10C). The “y” ions are those that include the C-terminus, the “b” ions are those that include the N-terminus, the inset at the right shows a match with the sequence of chitinase-3-like protein, and the inset above shows the intensity of the 117 reporter peak was 4.6 times that of the 114 peak. This and other proteins elevated in the NTHi treated mice are listed in Table 1.

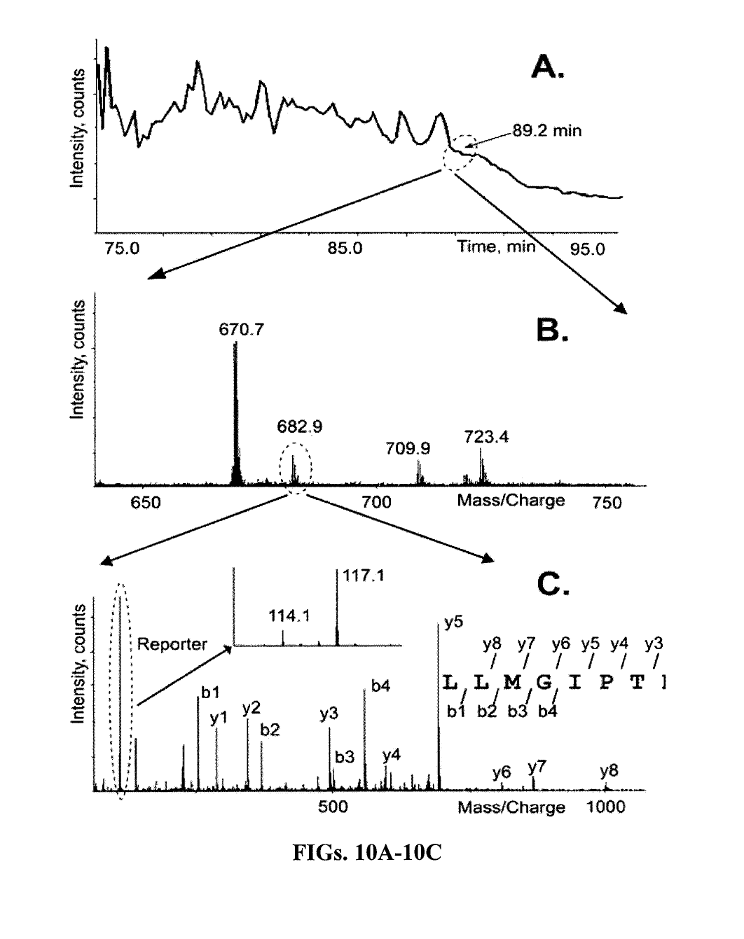

FIG. 11 Inflammatory Cell Counts in Bronchoalveolar Lavage Fluid of Mice Pretreated to Reduce Neutrophils then Treated with NTHi Lysate. Mice in groups of six each were pretreated with regimens listed in Table 1 to reduce neutrophil recruitment to the lungs. They were then exposed for 20 min to the aerosolized NTHi lysate, and 24 hr later BAL fluid was recovered and inflammatory cells counted as in FIG. 3. Shown are the mean ±SEM of the cell counts.

FIG. 12 Survival of Mice Deficient in Alveolar Macrophages and Neutrophils but Treated with NTHi Lysate, then Challenged with Spn Aerosol. The same data as those illustrated in FIG. 9 are shown here as a function of time to illustrate the delayed time to death in M/N deficient mice not protected by NTHi treatment.

FIG. 13 Survival rates of mice challenged with Spn and previously treated with NTHi at several time points. 100% survival resulted when mice received NTHi at 4, 8 h and 1 day before Spn challenge (Inset: shows these time points in more detail); 80-85% survival if received at 2 h, 2 and 3 days; 20% at 5 days; and 0% in the control group (PBS treated prior to Spn challenge). 6 mice per time point were infected to follow death rate.

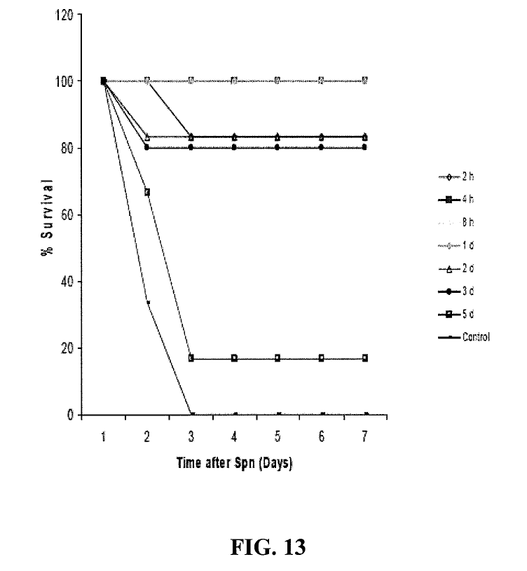

FIG. 14 Survival of Swiss-Webster mice treated with a 30-minute aerosol dose ALIIS 24 hr prior to Challenge with 5 LD₅₀ B. anthracis Ames spores.

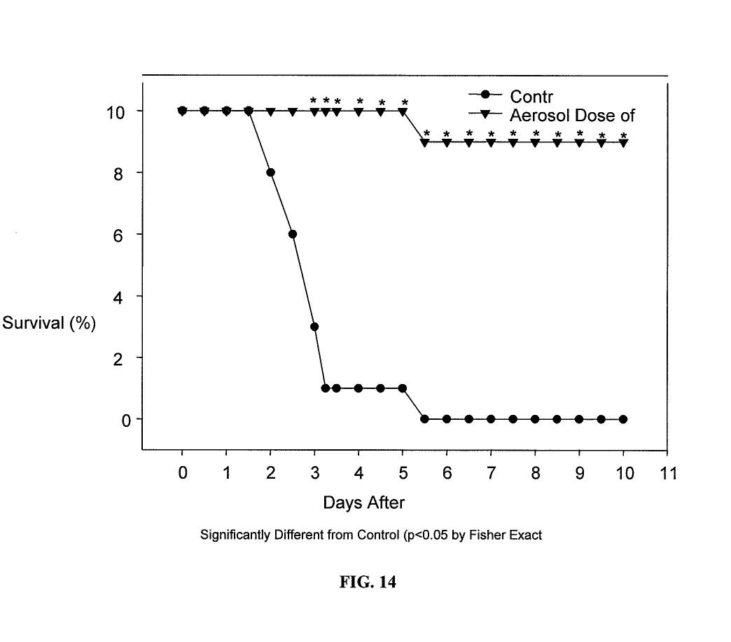

FIG. 15 Survival of Swiss-Webster mice immunized with ALIIS 24 hr prior to challenge with various doses of Y. pestis.

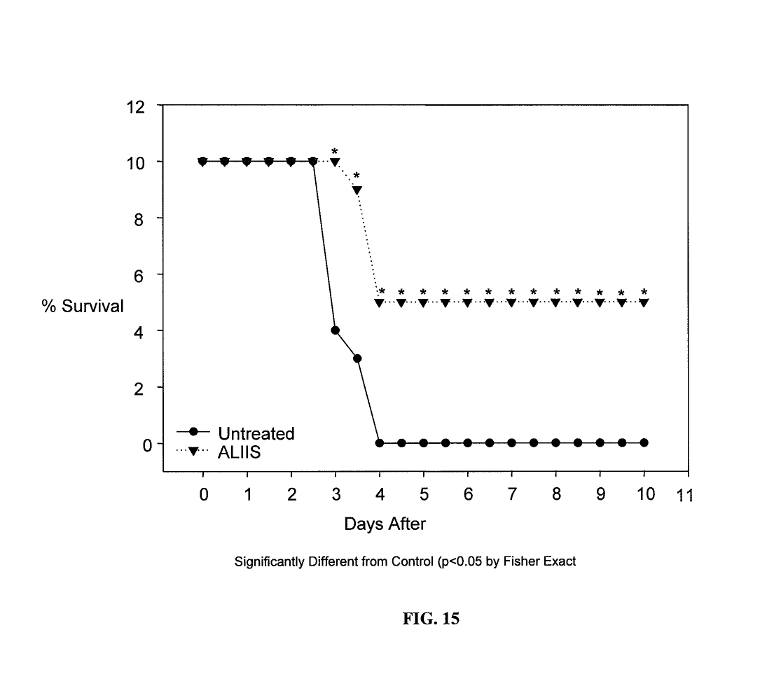

### DETAILED DESCRIPTION OF THE INVENTION

In response to certain inflammatory stimuli, the secretory cells of the airway epithelium of mice and humans rapidly undergo a remarkable change in structure termed inflammatory metaplasia. Most of the structural changes can be ascribed to increased production of secreted, gel-forming mucins, while additional macromolecules with functions in mucin secretion, microbial killing or inflammatory signaling are also upregulated. The physiologic function of this response is thought to be augmentation of local defenses against microbial pathogens, although that hypothesis has received only limited formal testing. Paradoxically, excessive production and secretion of gel-forming mucins is a major cause of airflow obstruction in common inflammatory diseases of the airways such as asthma, cystic fibrosis, and chronic obstructive pulmonary disease (COPD). The stimulation of the innate immunity without the production of mucin would provide an additional method of attenuating infection of the respiratory tract by preventing and/or treating a subject.

Embodiments of the invention include the stimulation of the airways of a subject with a microbial lysate including, but not limited to a microbial lysate comprising a killed microorganism. A microorganism may be killed by using a variety of methods known in the art, including, but not limited to sonication, irradiation, and the like. A subject administered the microbial lysate of the invention is afford a therapeutic, prophylactic, or therapeutic and prophylactic response to a potentially infecting organism. In particular aspects the microbial lysate is aerosolized and administered via the respiratory tract. The microbial lysate is used to induce or otherwise elicit a protective effect by, for example, activating or augmenting innate immunity of the lungs.

Embodiments of the invention include compositions comprising one or more bacterial lysate. Aspects of the invention include lysates derived from microorganism or a strain of the microorganism with a limited propensity for an infection that results in a disease state or, in the least, results in a disease state that rarely results in death or disability, i.e., results in limited effects, or results in little or no substantial morbidity or mortality. In certain aspects the microorganism is has a limited propensity to infect the lungs. Further aspects include aerosolized lysate of UV-killed non-typeable Haemophilus influenzae (NTHi) that can be used to elicit such a protective affect. In still further aspects, the microbial lysate does not cause an increased production of secreted mucins. Embodiments of the invention can be used as a preventive and preemptive therapeutic against for example, bioweapons, neo-virulent microbes, or opportunistic microbes.

### II. Stimulation of Lung Defenses

The inventors have used the mouse as model for microbial infection of the lung. In certain studies, untreated mice had mortality of 100%, but treated mice were highly protected. Protection was 100% for pretreatment 4 to 24 h before challenge, >80% for pretreatment 2 to 72 h before challenge, and substantial even when given 2 h after challenge. Protection does not depend on recruitment of neutrophils because mice made neutropenic with monoclonal antibody or cytosine arabinoside were still protected by the NTHi lysate. Protection is due to activation of local defenses since there was no protection from challenge with pneumococci given intravenously or intraperitoneally. Protection was associated with rapid bacterial killing, and proteomic analysis showed increased levels in bronchoalveolar lavage fluid of 25 proteins with putative antimicrobial activity, including lactoferrin, lysozyme, cathelicidins, and collectins. Typically, protection was also associated with increases in the inflammatory cytokines TNF-α, IFN-γ, and IL-6.

The effects of single and repetitive exposure of a subject to a composition of the invention have been determined and no obvious gross pathology, such as premature death, weight loss, or behavioral changes have been observed. During the first week of exposure to the inventive compositions, most inflammatory cells seen histopathologically are neutrophils, and these are observed predominantly around airways and blood vessels, with only few inflammatory cells in alveoli. Over time, there is a progressive increase in mononuclear cell infiltration around airway walls comprised predominantly of macrophages, B-cells, and CD8+ T-cells, which is ascribed to development of an adaptive immune response. Typically, no structural changes, such as mucous metaplasia, peribronchiolar fibrosis, or alveolar enlargement are observed in the lungs. However, after prolonged exposure, greater than 25 weeks, airway wall fibrosis may be seen.

Preclinical studies have been conducted to define the efficacy, mechanism, and toxicity of a composition and related methods of the invention. One benefit of the present invention is that it can be delivered and have effect quickly and easily. Also, the compositions can be produced economically in large quantities and easily stored, as well as easily transported by a person outside of a hospital setting. Typically, the administration of the inventive compositions and the methods of the invention result in at least some killing of the invading pathogens even before cellular entry. In the case that some pathogens do enter cells in the lungs either by escaping extracellular killing or because the compositions are administered after pathogen exposure (preemptively) instead of before pathogen exposure (preventatively), it is contemplated that the compositions and related methods promote intracellular killing resulting from the enhanced or augmented local responses in the lungs. The compositions and related methods are contemplated to have or produce protective or therapeutic responses against a variety of respiratory pathogens.

The protection or therapy afforded an individual by one type of microbial lysate, e.g., lysate produced from a non-pathogenic microorganism, may be extended to additional classes of microbial pathogens including gram negative bacteria, intracellular bacteria, fungi, and viruses because of the broad activity of innate antimicrobial mechanisms of the respiratory tract. An agent such as that described in this application would simplify countermeasure stockpiling and deployment. Also, the compositions and methods of the invention would eliminate the difficulty of rapidly identifying a specific pathogen during a bioweapon attack or other exposure or potential exposure event. In addition, the economic advantages of producing and purchasing an agent with applicability in multiple civilian and biodefense settings are significant. Augmenting local epithelial innate immune mechanisms is particularly attractive in subjects who often have neutropenia or impaired adaptive immune function, e.g., immune compromised subjects. The methods typically act locally rather than systemically, and provide broad effects against multiple pathogens. The effects are rapid and are attractive in a biodefense and epidemic setting.

Augmentation of innate defense capabilities of the lungs in normal hosts would be valuable during influenza or emergent respiratory viral epidemics for which adaptive immune vaccines are not available. Bacterial outbreaks with emergent or drug-resistant organisms might also be a situation in which boosting innate lung defenses could be helpful. Similarly, protection of caregivers during an epidemic would facilitate care of the sick while limiting spread.

Many people in the community live with chronically compromised defenses against infection, such as patients with diabetes and patients taking immunosuppressive drugs for autoimmune diseases or to prevent transplant rejection. These people might particularly benefit from augmentation of innate lung defenses during epidemics. Even more strikingly, cancer patients undergoing chemotherapy who have transient but severe compromise of immune defenses might benefit from transient protection. Pneumonia is a common occurrence in these patients, and is the leading cause of infectious death. Many chemotherapy drugs, such as alkylating agents and nucleoside analogs, cause severe transient neutropenia. Initially, neutropenic patients are susceptible to bacterial pneumonia from organisms seen in normal hosts, as well as bacteria of low virulence such as Stenotrophomonas maltophilia. With prolonged neutropenia, patients also become susceptible to infection with fungi of low virulence, particularly Aspergillus species. Innate immune defenses of the lung can be stimulated to provide transient protection during prolonged periods of neutropenia. Other cancer patients, such as those receiving fludarabine or anti-lymphocyte antibodies, or those receiving calcineurin inhibitors and steroids after hematopoietic stem cell transplantation, have impaired adaptive immunity. These patients might also benefit from episodic stimulation of lung innate immunity to protect against invasion by fungi and bacteria that have colonized the airways, or to protect against epidemic viruses. Community outbreaks of seasonal respiratory “cold” viruses such as parainfluenza and RSV can cause fatal pneumonia in these compromised patients, and infection with many of these viruses can be rapidly identified from nasal washings.

The immune system is the system of specialized cells and organs that protect an organism from outside biological influences. When the immune system is functioning properly, it protects the body against bacteria and viral infections, destroying cancer cells and foreign substances. If the immune system weakens, its ability to defend the body also weakens, allowing pathogens to grow in the body.

The immune system is often divided into: (a) an innate immunity comprised of components that provide an immediate “first-line” of defense to continuously ward off pathogens and (b) an adaptive (acquired) immunity comprising the manufacture of antibodies and production or stimulation of T-cells specifically designed to target particular pathogens. Using adaptive immunity the body can develop over time a specific immunity to particular pathogen(s). This response takes days to develop, and so is not effective at preventing an initial invasion, but it will normally prevent any subsequent infection, and also aids in clearing up longer-lasting infections.

Most multicellular organisms possess an “innate immune system” that does not change during the lifetime of the organism. In contrast, adaptive immunity is the responses to pathogens that change and develop during the lifetime of an individual. Organisms that possess an adaptive immunity also possess an innate immunity, and with many of the mechanisms between the systems being common, it is not always possible to draw a hard and fast boundary between the individual components involved in each, despite the clear difference in operation. Higher vertebrates and all mammals have both an innate and an adaptive immune system.

A. Innate Immune System.

The adaptive immune system may take days or weeks after an initial infection to have an effect. However, most organisms are under constant assault from pathogens that must be kept in check by the faster-acting innate immune system. Innate immunity defends against pathogens by rapid responses coordinated through “innate” mechanisms that recognize a wide spectrum of conserved pathogenic components. Plants and many lower animals do not possess an adaptive immune system, and rely instead on their innate immunity. Substances of both microbial and non-microbial sources are able to stimulate innate immune responses

The innate immune system, when activated, has a wide array of effector cells and mechanisms. There are several different types of phagocytic cells, which ingest and destroy invading pathogens. The most common phagocytes are neutrophils, macrophages, and dendritic cells. Another cell type, natural killer cells are especially adept at destroying cells infected with viruses. Another component of the innate immune system is known as the complement system. Complement proteins are normally inactive components of the blood. However, when activated by the recognition of a pathogen or antibody, the various proteins are activated to recruit inflammatory cells, coat pathogens to make them more easily phagocytosed, and to make destructive pores in the surfaces of pathogens.

The “first-line” defense includes physical and chemical barriers to infection, such as skin and mucus coating of the gut and airways, physically preventing the interaction between the host and the pathogen. Pathogens, which penetrate these barriers, encounter constitutively-expressed anti-microbial molecules (e.g., lysozyme) that restrict the infection. The “second-line” defense includes phagocytic cells (macrophages and neutrophil granulocytes) that can engulf (phagocytose) foreign substances.

Phagocytosis involves chemotaxis, where phagocytic cells are attracted to microorganisms by means of chemotactic chemicals such as microbial products, complement, damaged cells and white blood cell fragments. Chemotaxis is followed by adhesion, where the phagocyte sticks to the microorganism. Adhesion is enhanced by opsonization, where proteins like opsonins are coated on the surface of the bacterium. This is followed by ingestion, in which the phagocyte extends projections, forming pseudopods that engulf the foreign organism. Finally, the pathogen is digested by the enzymes in the lysosome, involving reactive oxygen species and proteases.

In addition, anti-microbial proteins may be activated if a pathogen passes through a physical barrier. There are several classes of antimicrobial proteins, such as acute phase proteins (e.g., C-reactive protein, which enhances phagocytosis and activates complement when it binds the C-protein of S. pneumoniae), lysozyme, and the complement system).

The complement system is a very complex group of serum proteins, which is activated in a cascade fashion. Three different pathways are involved in complement activation: (a) a classical pathway that recognizes antigen-antibody complexes, (b) an alternative pathway that spontaneously activates on contact with pathogenic cell surfaces, and (c) a mannose-binding lectin pathway that recognizes mannose sugars, which tend to appear only on pathogenic cell surfaces. A cascade of protein activity follows complement activation; this cascade can result in a variety of effects, including opsonization of the pathogen, destruction of the pathogen by the formation and activation of the membrane attack complex, and inflammation.

Interferons are also anti-microbial proteins. These molecules are proteins that are secreted by virus-infected cells. These proteins then diffuse rapidly to neighboring cells, inducing the cells to inhibit the spread of the viral infection. Essentially, these anti-microbial proteins act to prevent the cell-to-cell proliferation of viruses.

B. Adaptive Immune System

The adaptive immune system, also called the “acquired immune system,” ensures that most mammals that survive an initial infection by a pathogen are generally immune to further illness, caused by that same pathogen. The adaptive immune system is based on dedicated immune cells termed leukocytes (white blood cells) that are produced by stem cells in the bone marrow, and mature in the thymus and/or lymph nodes. In many species, including mammals, the adaptive immune system can be divided into: (a) a humoral immune system that acts against bacteria and viruses in the body liquids (e.g., blood) by means of proteins, called immunoglobulins (also known as antibodies), which are produced by B cells; and (b) a cellular immune system that destroys virus-infected cells (among other duties) with T cells (also called “T lymphocytes”; “T” means they develop in the thymus). The adaptive immune system is typically directed toward a specific pathogen, e.g., vaccination.

### II. Microbial Lysates

Typically, a non-pathogenic microorganism can be grown in vitro, harvested and prepared as a lysate by various methods. Methods of producing a lysate are know in the art, for instance, typically a microorganism is grown under conditions established for its growth. The microorganisms are then harvested, typically by centrifugation, filtration, and the like. After being harvested the microorganism is washed and resuspended in an appropriate buffer. This suspension is typically treated to kill the organism, typically by UV irradiation, physical or chemical methods. Typically the suspension can be killed and/or physically disrupted by mechanical and non-mechanical methods, such as sonication, emulsification/homogenization, biological (e.g., viral lysis), barometric, pneumatic (e.g., hypotonicity), detergent, alkali, and/or enzymatic methods, e.g., using a Sonic Dismembrator 50 (Fisher Scientific International Inc., Hampton, N.H.; or EmulsiFlex™ homogenizer (Avestin Inc., Ottawa, Canada). In certain embodiments a homomogenizer can be used to emulsify a microbe composition. For example, a homogenizer can be air/gas driven, and have a high pressure pump. The pump can be operatively coupled to a homogenizing valve, such as a pneumatically controlled, dynamic homogenizing valve. The microbe composition can be put under an appropriate capacity/pressure. The flow rate depends on the homogenizing pressure selected. The homogenizing pressure can be adjusted to an appropriate level or range, for instance the EmulsiFlex™, by Avestin Inc., Ottawa, Canada, has the range of 500-30000 psi/3.5-207 MPa. Inlet and outlet temperatures can be controlled with installation of an appropriate heat exchanger. Most laboratories, research facilities and production spaces have sufficient air pressure and flow rate to run such a emulsifier/homogenizer. In certain aspects, a nitrogen gas cylinder or small compressor of 3hp/2.2 kW is sufficient. The air/gas pressure required depends on the application. For most dispersions, emulsions, liposomes and bacterial rupture, an air pressure of 85 psi/0.6 MPa or more is sufficient.

The lysate is then quantitated for protein or other components and adjusted to an appropriate level for administration. The lysate is then prepare for delivery, typically by loading in a device for aersolization, e.g., suspension is placed in an AeroMist CA-209 nebulizer driven by 10 l/min of room air supplemented with 5% CO₂. Aspects of the invention can be used with fungi, virus, bacteria and other microorganisms. Growth and harvesting of these organisms is typically know in the art.

This lysate can be formulated into a pharmaceutically acceptable composition for administration to subject in need of such treatment or administration. Bacterial, viral, and/or fungal strains can be obtained from various vendors, which include the American Type Culture Collection (Manassas, Va.), United States Government, and the like. A microorganism will be grown on or in a particular medium or cell type that is typically well known to those of skill in virology, microbiology, mycology, or medicine. For example, NTHi strain of bacteria are typically grown on chocolate agar plates (Remel Inc.) for 24 hr at 37° C. in a 5% CO₂ atmosphere, then harvested and incubated for 16 hr under the same conditions in brain-heart infusion broth (Acumedia Manufacturers, Inc., Baltimore, Md.) supplemented with NAD 3.5 μg/ml.

Microorganisms may then be harvested, for example a bacterial culture can harvested by centrifugation. Harvested microorganisms are typically washed and suspended in an appropriate solution or buffer, e.g., PBS. The microorganisms are then treated, i.e., lysed, for example, by extraction, irradiation, sonication, homogenization, rapid freeze-thaw, osmotic shock, etc. or combinations of these treatments to degrade the microorganisms into various non-viable components. Typically the protein concentration is adjusted to an appropriate concentration in PBS or another pharmaceutically acceptable solution. The microbial lysates can then be formulated or manipulated for delivery to the respiratory tract, e.g., by aerosolization or nebulization.

A variety of microorganisms can be used to produce the microbial lysates. Microorganisms include viruses, bacteria, and fungi. Typically these microorganisms will be classified as non-virulent or non-pathogenic microorganisms in order to limit any adverse effects of a fraction of viable microorganisms that may be present in the microbial lysates. Microorganisms deemed non-pathogenic or of limited pathogenicity include, but is not limited to:

Bacteria—Acetobacter aceti, Bacillus cereus, B. licheniformis, B. megaterium, B. pumilus, B. subtilis, Erwinia dissolvens, Lactobacillus acidophilus, L. bulgaricus, L. casei, L. delbrückii, L. helveticus, L. lactis, Leuconostoc, L. mesenteroides, Pediococcus, Propionibacterium acidipropionici, P. freundenreichii, P. jensenii, P. shermanii, P. technicum, P. thoenii, Streptococcus cremoris, S. diacetilactis, S. faecalis, S. lactis, or S. thermophilus.

Fungi—Penicillium camemberti, P. roqueforti, Rhodotorula rubrum, Saccharomyces cerevisiae, Basidiomycetes, Dactylaris, Deuteromycetes, Taxomyces andreanae, Zygomycetes or S. uvarum.

Green algae—all photosynthetic forms except Prototheca, including, but not limited to Ankistrodesmus, Bangia, Batrachospermum, Bulbochaete, Callithamnion, Careria, Caulerpa, Chlamydomonas, Chlorella, Cladophora, Closterium, Coccolithophora, Corallina, Cosmarium, Derbesia, Desmids, Dunaliella, Dictyota, Ectocarpus, Egregia, Enteromorpha, Eremosphaera, Eudorina, Fritschiella, Fucus, Gigartina, Gonium, Gracilaria, Hydrodictyon, Iridea, Laminaria, Macrocystis, Mesotaenium, Micrasterias, Microspora, Mougeotia, Nereocystis, Netrium, Nitella, Ochromonas, Oedogonium, Pandorina, Pediastrum, Polysiphonia, Porphyra, Porphyridium, Protococcus, Scenedesmus, Selanastrum, Spirogyra, Staurastrum, Stigeoclonium, Synura, Tribonema, Ulothrix, Ulva, Vaucheria, Volvox, or Zygnema.

Protozoa: Achnanthes, Actinosphaerium, Amoeba proteus, Amoeba chaos, Amphidinium, Arcella, Astasia, Difflugia, Blepharisma, Bursaria truncatella, Chilomonas, Colpidium, Crithidia fasciulata, Cyclotella, Didinium, Euglena, Euplotes, Gregarines, Herpetomonas muscarum, Leishmania tarentalae, Leptomonas pessoai, Navicula, Paramecium, Peranema, Peridinium, Phacus, Prorocentrum, Pyrsonympha, Spirostomum, Stentor, Synedra, Tetrahymena, Thalassiosira, Trachelomonas, Tritrichomonas augusta, Trypanosoma lewisi, Trypanosoma ranarum, Trichonympha, or Vorticella.

Lichens—All forms.

Slime Molds: All types, including Dictyostelium and Physarum.

Viruses—Coliphages, bacteriophages (except those that confer pathogenicity to Corynebacterium diphtheriae), or to otherwise non-pathogenic bacteria; Abelson murine leukemia virus; Aviadenovirus; Baculovirus; Border disease virus; Bovine viral diarrhea virus; Canine distemper virus; Canine parvovirus; Capripoxvirus; Epizootic hemorhhagic disease virus; Equine herpes virus type-I; Equine infectious anemia virus; Equine influenza virus; Feline panleukopenia virus; H-1 virus; Haemophilus paragallinarum; Herpesvirus salmonis; Ictalurid herpesvirus 1; Infectious bursal disease virus; Minute virus of mice; Murine leukemia virus; Myxoma virus; Pneumonia virus of mice; Porcine parvovirus; Porcine respiratory coronavirus; Porcine transmissible gasteroenteritis virus; or Rat cytomegalovirus.

Archaebacteria—all free-living species, such as Halobacterium salinarium, Halococcus agglomeratus, and Methanomonas methylovora.

Cyanobacteria—Anabaena, Anacystis, Cyanophora, Cylindrospermum, Fischerella, Glaucocystis, Gloeocapsa, Gloeotrichia, Lyngbya, Merismopedia, Nostoc, Oscillatoria, Scytonema, Spirulina, or Tolypothrix.

In a particular aspect the NTHi strain of Haemophilus or E. coli can be used to produce a lysate of the invention.

### III. Potentially Pathogenic Organisms

Embodiments of the invention include compositions and related methods for a broad protection against a variety of pathogens or potential pathogens. For example, bacterial pneumonia in a normal host occurs at a rate of 1/100 persons/year, mostly in elderly adults and young children and can be caused by a variety of organisms. It is most commonly caused by Streptococcus pneumoniae, followed in frequency by encapsulated Hemophilus influenzae. Other bacteria such as enteric gram negatives, anaerobes, and Staphylococcus aureus are significant causes of pneumonia in specific settings, such as healthcare facilities. Mycobacterium tuberculosis is highly infectious, and historically was an important cause of mortality worldwide. It has mostly been controlled with antibiotics in the developed world, though multidrug-resistant strains continue to cause problems and are classified as Category C bioweapon agents. Legionella pneumophila was first identified during an outbreak in Philadelphia in 1978, though it is now recognized to occur widely at a low endemic rate related to environmental sources. Also, fungal infections of the lungs can cause symptomatic disease in normal hosts. Histoplasma capsulatum, Coccidiodes immitis, Blastomyces dermatitidis, and Crytococcus neoformans can all cause pneumonia related to local exposure to high environmental concentrations. Pneumonia due to these pathogenic fungi is usually self-limited in normal hosts. Some additional “atypical” microorganisms, such as mycoplasmas, account for a substantial fraction of additional pneumonias in normal hosts. It is contemplated that a composition of the present invention can provide a rapid, temporal protection against a spectrum of agents that can cause, for example pneumonia or other disease states. In certain aspects the present invention may be used in combination with a vaccination regime to provide an additional protection to a subject that may or is exposed to one or more pathogenic or potentially pathogenic organism.

In particular aspects of the invention the compositions and methods of the invention may be used to prevent, reduce the risk of or the treat infection or exposure to a biological weapon or intentional exposure of a subject(s) to an inhaled infective agent. The only microbial pathogen that has been used as a terrorist weapon in the modern era is Bacillus anthracis, which has a case-fatality rate of 75% when infection occurs by the respiratory route, even with the use of appropriate antibiotics. Francisella tularensis is an aerobic, gram negative coccobacillus that is a facultative intracellular pathogen. It is highly infectious, highly pathogenic, and survives under harsh environmental conditions, making it a serious bioterror threat even though it is poorly transmissible from person to person (Dennis, 2001). A vaccine is available, but is only partially protective. The World Health Organization estimated that aerosol dispersal of 50 kg of virulent Francisella tularensis over a metropolitan area with 5 million inhabitants would result in 250,000 incapacitating casualties, including 19,000 deaths; the Centers for DiseaseCDC estimated the economic cost of such an attack to be $5.4 billion for every 100,000 persons exposed (Dennis, 2001). Other Class A bioterrorism agents that can be transmitted by aerosol are Yersinia pestis, smallpox virus, and hemorrhagic fever viruses. In addition, multiple Class B and C agents can be effectively delivered by the respiratory route. Together, these organisms comprise gram-positive, gram-negative, intracellular, and extracellular bacteria, as well as a variety of viral classes. Because of the potential difficulty in initially identifying a specific bioterrorism agent, the complexity of locally stockpiling adaptive immune vaccines and antibiotics directed at specific agents, and the remarkable virulence of organisms such as Bacillus anthracis despite appropriate treatment, stimulation of innate defense capabilities of the lungs that could either prevent or preempt infection with a bioterror agent delivered by the respiratory route could have great public health value.

A. Pathogenic or Potentially Pathogenic Bacteria

There are numerous bacterial species that are considered pathogenic or potentially pathogenic under certain conditions. In certain aspect, the pathogenicity is determined relative to infection via the lungs. These bacteria include, but are not limited to various species of the Bacillus, Yersinia, Franscisella, Streptococcus, Staphylococcus, Pseudomonas, Mycobacterium, Burkholderia genus of bacteria. Particular species of bacteria from which a subject may be protected include, but is not limited to Bacillus anthracis, Yersinia pestis, Francisella tularensis, Streptococcus pnemoniae, Staphylococcus aureas, Pseudomonas aeruginosa, Burkholderia cepacia, Corynebacterium diphtheriae, Clostridia spp, Shigella spp., Mycobacterium avium, M. intracellulare, M. kansasii, M. paratuberculosis, M. scrofulaceum, M. simiae, M. habana, M. interjectum, M. xenopi, M. heckeshornense, M. szulgai, M. fortuitum, M. immunogenum, M. chelonae, M. marinum, M. genavense, M. haemophilum, M. celatum, M. conspicuum, M. malmoense, M. ulcerans, M. smegmatis, M. wolinskyi, M. goodji, M. thermoresistible, M. neoaurum, M. vaccae, M. palustre, M. elephantis, M. bohemicam and M. septicum.

B. Virus

There are numerous virus and viral strains that are considered pathogenic or potentially pathogenic under certain conditions. Viruses can be placed in one of the seven following groups: Group I: double-stranded DNA viruses, Group II: single-stranded DNA viruses, Group III: double-stranded RNA viruses, Group IV: positive-sense single-stranded RNA viruses, Group V: negative-sense single-stranded RNA viruses, Group VI: reverse transcribing Diploid single-stranded RNA viruses, Group VII: reverse transcribing Circular double-stranded DNA viruses. Viruses include the family Adenoviridae, Arenaviridae, Caliciviridae, Coronaviridae, Filoviridae, Flaviviridae, Hepadnaviridae, Herpesviridae (Alphaherpesvirinae, Betaherpesvirinae, Gammaherpesvirinae), Nidovirales, Papillomaviridae, Paramyxoviridae (Paramyxovirinae, Pneumovirinae), Parvoviridae (Parvovirinae, Picornaviridae), Poxyiridae (Chordopoxyirinae), Reoviridae, Retroviridae (Orthoretrovirinae), and/or Togaviridae. These virus include, but are not limited to various strains of influenza, such as avian flu (e.g., H5N1). Particular virus from which a subject may be protected include, but is not limited to Cytomegalovirus, Respiratory syncytial virus and the like.

Examples of pathogenic virus include, but are not limited to Influenza A, H5N1, Marburg, Ebola, Dengue, Severe acute respiratory syndrome coronavirus, Yellow fever virus, Human respiratory syncytial virus, Vaccinia virus and the like.

C. Fungus

There are numerous fungal species that are considered pathogenic or potentially pathogenic under certain conditions. Protection can be provided for, but not limited to Aspergillus fumigatus, Candida albicans, Cryptococcus neoformans, Histoplasma capsulatum, Coccidioides immitis, or Pneumocystis carinii, and/or Blastomyces dermatitidis.

### IV. Formulations and Administration

The pharmaceutical compositions disclosed herein may be administered via the respiratory system of a subject. Microbial lysates may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations contain a preservative to prevent the growth of microorganisms. The pharmaceutical forms suitable for inhalation include sterile aqueous solutions or dispersions and sterile powders for the extemporaneous preparation of sterile inhalable solutions or dispersions. In all cases the form must be sterile and must be capable of inhalation directly or through some intermediary process. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

Sterile compositions are prepared by incorporating the active components in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some methods of preparation are vacuum-drying and freeze-drying techniques which yield a powder of the active ingredient plus any additional desired ingredient from a previously sterile-filtered solution thereof.

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258, 6,794,357, 6,737,045, and 6,488,953, all of which are incorporated by reference. According to the invention, at least one pharmaceutical composition can be delivered by any of a variety of inhalation or nasal devices known in the art for administration of a therapeutic agent by inhalation. Other devices suitable for directing pulmonary or nasal administration are also known in the art. Typically, for pulmonary administration, at least one pharmaceutical composition is delivered in a particle size effective for reaching the lower airways of the lung or sinuses.

All such inhalation devices can be used for the administration of a pharmaceutical composition in an aerosol. Such aerosols may comprise either solutions (both aqueous and non aqueous) or solid particles. Metered dose inhalers typically use a propellant gas and require actuation during inspiration. See, e.g., WO 98/35888; WO 94/16970. Dry powder inhalers use breath-actuation of a mixed powder. See U.S. Pat. Nos. 5,458,135; 4,668,218; PCT publications WO 97/25086; WO 94/08552; WO 94/06498; and European application EP 0237507, each of which is incorporated herein by reference in their entirety. Nebulizers produce aerosols from solutions, while metered dose inhalers, dry powder inhalers, and the like generate small particle aerosols. Suitable formulations for administration include, but are not limited to nasal spray or nasal drops, and may include aqueous or oily solutions of the microbial lysate.

A spray comprising a pharmaceutical composition of the present invention can be produced by forcing a suspension or solution of a composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

A pharmaceutical composition of the present invention can be administered by a nebulizer such as a jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the composition creating an aerosol.

In a metered dose inhaler (MDI), a propellant, a composition, and any excipients or other additives are contained in a canister as a mixture with a compressed gas. Actuation of the metering valve releases the mixture as an aerosol.

Pharmaceutical compositions for use with a metered-dose inhaler device will generally include a finely divided powder containing a composition of the invention as a suspension in a non-aqueous medium, for example, suspended in a propellant with the aid of a surfactant. The propellant can be any conventional material employed for this purpose such as chlorofluorocarbon, a hydrochlorofluorocarbon, a hydrofluorocarbon, or a hydrocarbon including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art.

### V. Combination Treatments

The compositions and methods of the present invention may be used in the context of a number of therapeutic or prophylactic applications. In order to increase the effectiveness of a treatment with the compositions of the present invention, e.g., microbial lysates, or to augment the protection of another therapy (second therapy), e.g., vaccination or antibiotic therapy, it may be desirable to combine these compositions and methods with other agents and methods effective in the treatment, reduction of risk of infection, or prevention of diseases and pathologic conditions, for example, anti-bacterial, anti-viral, and/or anti-fungal treatments.

Various combinations may be employed; for example, a microbial lysate, such as NTHi lysate, is “A” and the secondary therapy is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the microbial lysate of the present invention to a subject will follow general protocols for the administration via the respiratory system, and the general protocols for the administration of a particular secondary therapy will also be followed, taking into account the toxicity, if any, of the treatment. It is expected that the treatment cycles would be repeated as necessary. It also is contemplated that various standard therapies, as well as vaccination, may be applied in combination with the described therapies.

In certain aspects of the invention an anti-inflammatory agent may be used in combination with a microbial lysate.

Steroidal anti-inflammatories for use herein include, but are not limited to fluticasone, beclomethasone, any pharmaceutically acceptable derivative thereof, and any combination thereof. As used herein, a pharmaceutically acceptable derivative includes any salt, ester, enol ether, enol ester, acid, base, solvate or hydrate thereof. Such derivatives may be prepared by those of skill in the art using known methods for such derivatization.

Fluticasone—Fluticasone propionate is a synthetic corticosteroid and has the empirical formula C₂₅H₃₁F₃O₅S. It has the chemical name S-(fluoromethyl)6α,9-difluoro-11β17-dihydroxy-16α-methyl-3-oxoandrosta-1,4-diene-17β-carbothioate,17-propionate. Fluticasone propionate is a white to off-white powder with a molecular weight of 500.6 and is practically insoluble in water, freely soluble in dimethyl sulfoxide and dimethylformamide, and slightly soluble in methanol and 95% ethanol.

In an embodiment, the formulations of the present invention may comprise a steroidal anti-inflammatory (e.g., fluticasone propionate)

Beclomethasone—In certain aspects the steroidal anti-inflammatory can be beclomethasone dipropionate or its monohydrate. Beclomethasone dipropionate has the chemical name 9-chloro-11b,17,21-trihydroxy-16b-methylpregna-1,4-diene-3,20-doine 17,21-dipropionate. The compound may be a white powder with a molecular weight of 521.25; and is very slightly soluble in water (Physicians' Desk Reference), very soluble in chloroform, and freely soluble in acetone and in alcohol.

Providing steroidal anti-inflammatories according to the present invention may enhance the compositions and methods of the invention by, for example, attenuating any unwanted inflammation. Examples of other steroidal anti-inflammatories for use herein include, but are not limited to, betamethasone, triamcinolone, dexamethasone, prednisone, mometasone, flunisolide and budesonide.

In accordance with yet another aspect of the invention, the non-steroidal anti-inflammatory agent may include aspirin, sodium salicylate, acetaminophen, phenacetin, ibuprofen, ketoprofen, indomethacin, flurbiprofen, diclofenac, naproxen, piroxicam, tebufelone, etodolac, nabumetone, tenidap, alcofenac, antipyrine, amimopyrine, dipyrone, animopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, meclofenamic acid, mefenamic acid, niflumic acid, salidifamides, sulindac, suprofen, tolmetin, nabumetone, tiaramide, proquazone, bufexamac, flumizole, tinoridine, timegadine, dapsone, diflunisal, benorylate, fosfosal, fenclofenac, etodolac, fentiazac, tilomisole, carprofen, fenbufen, oxaprozin, tiaprofenic acid, pirprofen, feprazone, piroxicam, sudoxicam, isoxicam, celecoxib, Vioxx® and tenoxicam.

### VI. KITS

Any of the compositions described herein may be comprised in a kit. In a non-limiting example, reagents for delivery of a microbial lysate are included in a kit. In certain aspects the kit is portable and may be carried on a person much like an asthma inhaler is carried. The kit may further include pathogen detector. The kit may also contain a gas or mechanical propellant for compositions of the invention.

The components of the kits may be packaged either in an aqueous, powdered or lyophilized form. The container means of the kits will generally include at least one inhaler, canister, vial, test tube, flask, bottle, syringe or other container means, into which a component may be placed, and preferably, suitably aliquoted. Where there is more than one component in the kit (antibiotic, second lysate, etc.), the kit also will generally contain a second, third or other additional container into which the additional components may be separately placed. However, various combinations of components may be comprised in a vial, canister, or inhaler. A container of the invention can include a canister or inhaler that can worn on a belt or easily carried in a pocket, backpack or other storage container. The kits of the present invention also will typically include a means for containing the microbial lysates, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred, but not required. However, the components of the kit may be provided as dried powder(s). When reagents and/or components are provided as a dry powder, the powder may be reconstituted by the addition of a suitable solvent or administered in a powdered form. It is envisioned that the solvent may also be provided in another container means.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used directly or indirectly in the detection of pathogenic microorganisms or administration of a microbial lysate of the invention.

### I. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Protective Microbial Lysates

### A. Material and Methods

Experimental animals. Female, specific pathogen free, 5-6 week old BALB/c mice were purchased from Harlan (Indianapolis, Ind.). Mice were housed and handled in accordance with the Institutional Animal Care and Use Committee of the MD Anderson Cancer Center. The number of survival studies was minimized in order to minimize animal discomfort.

Spn pneumonia challenge. One ml of frozen bacterial stock (1×10⁹ CFU) was incubated for 16 hr in 150 ml of Todd-Hewitt broth (Becton Dickinson Diagnostic Systems, Franklin Lakes, N.J.) at 37° C. in a 5% CO₂ atmosphere. This suspension was diluted in 1.5 l of fresh broth and grown for 6-7 hr in logarithmic phase to achieve an OD₆₀₀ of 0.25 to 0.40, which corresponded to bacterial counts of 1-2×10¹⁰ CFU. The suspension was then concentrated by centrifugation at 2500×g for 10 min at 4° C., the pellet was washed with 20 ml PBS, and resuspended in 15 ml PBS. The bacterial concentration was then determined by plating out 100-fold dilutions onto blood-agar plates (Remel Inc. Lenexa, Kans.). For nebulization, a 10 ml sample of the bacterial suspension was placed in a compressed gas nebulizer driven by room air supplemented with 5% CO₂ at a flow rate of 10 liters/min. A 10 ml sample of the final suspension was placed in an AeroMist CA-209 compressed gas nebulizer (CIS-US, Inc., Bedford, Mass.), driven by 101/min of room air supplemented with 5% CO₂ to promote maximal ventilation and homogeneous exposure throughout the lungs. After 30 min of aerosolization, an additional 5 ml of the suspension was added, and aerosolization continued for another 30 min. During the full hour, approximately 8 ml of suspension was aerosolized.

NTHi lysate treatment. NTHi were grown on chocolate agar plates (Remel Inc.) for 24 hr at 37° C. in a 5% CO₂ atmosphere, then harvested and incubated for 16 hr under the same conditions in brain-heart infusion broth (Acumedia Manufacturers, Inc., Baltimore, Md.) supplemented with NAD 3.5 μg/ml. The culture was centrifuged at 2500×g for 10 min at 4° C., washed and resuspended in phosphate-buffered saline solution (PBS) (Gibco, Invitrogen Corporation, Grand Island, N.Y.). This bacterial suspension was UV irradiated at 3000 μJ/cm² (UV Stratalinker 1800, Stratagene, Cedar Creek, Tex.) and typically sonicated three times for 30 sec (Sonic Dismembrator 50, Fisher Scientific International Inc., Hampton, N.H.). The final protein concentration was adjusted to 2.5 mg/ml in PBS. A 10 ml sample of the final suspension was placed in an AeroMist CA-209 nebulizer driven by 101/min of room air supplemented with 5% CO₂. A 20 min nebulizing period resulted in the utilization of approximately 4-6 ml of lysate, and the protein concentration in the residual lysate was measured at 2.5 mg/ml. The aerosol particles generated were measured using an Andersen cascade impacter (Andersen Instruments, Atlanta, Ga.) and ranged in size from <0.4 μm to 4.7 μm with a mass median aerodynamic diameter of 1.49 μm and a geometric SD of 1.91 μm. Endotoxin levels were measured using the PyroGene Assay kit, and purified E. coli endotoxin for aerosol treatment was dissolved in PBS (both from Cambrex). Alternatively, a microbial lysate can be generated by homogenization, e.g., emulsifying a microbial composition in a homogenizer such as an Emulsiflex homogenizer (Avestin, Inc.).

BAL and measurement of inflammatory cell exudates. Mice were anesthetized by intraperitoneal injection of a mixture of ketamine, xylazine and acepromazine, then tracheostomized using a sterile luer stub adapter cannula (Becton Dickinson Primary Care Diagnostics, Sparks, Md.). BAL fluid was obtained by sequentially instilling and collecting two aliquots of one ml each through the cannula. The total leukocyte count was determined using a hemacytometer (Hauser Scientific, Horsham, Pa.). Cell populations were determined by cytocentrifugation of 300 μl of BAL using a Cytospin 4 (Thermo Electron Corporation, Waltham, Mass.) at 2,000 rpm for 5 min, followed by Wright-Giemsa staining.

Histological analysis. For light microscopy studies, whole lungs were perfused with PBS via the right cardiac ventricle. Fixative (4% paraformaldehyde in 0.1 M phosphate buffer, pH 7.0) was infused intratracheally at 10-15 cm pressure. The lungs were first fixed in situ at room temperature, then removed from the thoracic cavity and further fixed overnight at 4° C. Fixed lungs were embedded in paraffin and processed for light microscopy using hematoxylin and eosin staining.

Measurement of bacterial counts in mouse lungs. Lungs were harvested from anesthetized mice, and then homogenized in 1 ml of PBS using a 2 ml tissue grinder (Kontes Glass Company, Vineland, N.J.). 100 μl aliquots of 10⁻⁴ and 10⁻⁶ dilutions were plated on blood agar (Remel, Inc., Lenexa Kans.), then incubated overnight at 37° C. in 5% CO₂. Colonies were counted and the numbers converted to CFU/ml of homogenate.

Depletion of neutrophils and alveolar macrophages (AM). Several agents were tested to induce neutropenia in mice prior to NTHi stimulation (day 0): RB6-8C5 monoclonal antibody (Becton Dickinson Biosciences Pharmingen, San Diego, Calif.) by iv injection of 50 μg/d at days −1 and 0; cytosine arabinoside (Sigma-Aldrich Inc, St Louis, Mich.) 300 mg/kg, ip injection at days −8, −5, −2 and −1 (scheme 1), and 300 mg/kg, ip, at −48 h, −24 h, −12 h and day 0 (scheme 2); busulphan (Fluka, Sigma-Aldrich Inc, St Louis, Mo.) 125 mg/kg, ip, at days −8, −5, −2, and −1 (scheme 1), and 125 mg/kg, ip, at days −11, −8, −6, −4, and −1 (scheme 2); 5-fluorouracil (Sigma-Aldrich Inc) 150 mg/kg, ip, at days −8, −5 −2 and −1 (scheme 1), and 150 mg/kg, ip, at days −8 and −3 (scheme 2); and cyclophosphamide (Sigma-Aldrich Inc) 200 mg/kg, ip, at days −5, −2, and −1. The efficacy of these depletion agents at different doses as well as their effects in survival were evaluated (supplement data). RB6-8C5 monoclonal antibody, administered previously to NTHi stimulation, depleted neutrophils in BAL without affecting survival, thus it was used for the following neutrophil depletion experiments. Control mice received iv injection of rat IgG at the same dose as RB6-8C5 antibody. For AM depletion, liposome-encapsulated clodronate was used. 100 μl of liposome-encapsulated clodronate, or liposome-encapsulated PBS as control was delivered intranasally 1, 2 and 3 days prior to infection with Spn.

Proteomic analysis. BAL fluid was centrifuged at 15000×g for 5 min and supernatants were collected, lyophilized, and resuspended in 150 μl ddH₂0. To remove salt prior to proteomic analysis samples were subjected to acetone precipitation at 4 volumes of cold acetone (−20° C.) to 1 volume of protein solution, spun at 15000×g, supernatant discarded and pellet resuspended in 100 μl ddH₂O, which brought the protein concentration to approximately 1 mg/ml, measured by modified Lowry assay (BCA protein assay reagent kit Cat No. 23225, Pierce, Rockford, Ill.).

SDS-PAGE, using precast gradient 4-15% Tris-HCL Ready Gel (Bio-Rad Laboratories, Hercules, Calif.), was utilized for resolution of BAL supernatant proteins. Gels were loaded with 20 μg of BAL protein, ran at 100 V for 2 hours and stained with Coomasie Blue-R250 (Bio-Rad Laboratories, Hercules, Calif.) for bands visualization.

High Performance Liquid Chromatography (HPLC) was performed on a Hewlett-Packard 1090 binary gradient HPLC (Agilent Technologies, Little Falls, Del.) on a 1 mm×25 cm C4 column (Vydac, Hesperia, Calif.) with UV detection. Water/acetonitrile gradients containing 0.1% trifluoroacetic acid were run at 120 μl/min and monitored by UV absorbance at both 214 nm and 295 nm. Fractions were collected by hand and reduced in volume by vacuum centrifugation for analysis. Proteins from HPLC fractions were digested 200 ng with sequencing-grade modified trypsin (Promega, Madison, Wis.) in 30 mM ammonium bicarbonate overnight at 37° C. The resulting peptides were analyzed by LC-MS/MS and identified by database searching (described below).

Electrospray mass spectrometry (ESI-MS) was performed on a Qq-TOF, quadrupole time-of-flight instrument (QStar-Pulsar-i, Applied Biosystems/MDS-Sciex, Foster City, Calif.). Electrospray ion trap mass spectrometry was performed on a linear ion-trap mass spectrometer (LTQ, Thermo-Finnigan, San Jose, Calif.). Proteins were identified by database searching against the non-redundant NCBI protein database using online database searching tool Mascot (Matrix Science, London, UK).

For the Quantitative Isobaric Stable Isotope Tag (iTRAQ, ref 41) experiment BAL precipitates were resuspended in 20 μl of reaction buffer (0.1% SDS, 20 mM PBS pH 8), reduced with 2 mM tris(2-carboxyethyl) phosphine and alkylated with 10 mM S-methyl methanethiosulfonate, digested overnight with trypsin, and separately derivatized with one each of the isobaric reagents. The control extract was labeled with iTRAQ114, and the stimulated extract was labeled with iTRAQ117 (all reagents from Applied Biosystems, Foster City, Calif.). The derivatized digests were combined and analyzed by LC-MS/MS on the Qstar-Pulsar-i. Data was analyzed either manually by database search and inspection of the spectra, or using ProQuant software (Applied Biosystems/MDS-Sciex, Foster City, Calif.).

For the Difference Gel Electrophoresis precipitated proteins were dissolved in denaturing lysis buffer (7M urea, 2M thiourea, 4% CHAPS, 1% Triton X-100, 10 mM dithiothreitol (DTT), 10 mM HEPES pH 8.0). Particulate matter was removed by centrifugation at 12,000×g for 15 minutes at 4° C. One hundred micrograms of each of two protein samples to be compared were labeled on lysine residues with either Cy3 or Cy5 fluorescent dyes (obtained through the Integrated Core Facility at the University of Pittsburgh). The labeled samples were loaded by rehydration or cup-loading into 24 cm Immobiline pH 3-10 Isoelectric Focusing (IEF) strips (Amersham Biosciences, Piscataway, N.J.). The first dimension gels were focused to 70,000 volt-hours on an Ettan IPGphor power source (Amersham Biosciences). Second dimension gels were 10% acrylamide and run on an EttanDalt Six apparatus (Amersham Biosciences). Images were acquired on a Typhoon scanner (Amersham), and downloaded into Image J, a freeware program available through NIH. Cy3 and Cy5 images were stacked, and a two-frame movie was evaluated visually for changes in spot intensity. At least two independent comparisons were performed to identify repeatable differences. Gels were post-stained with colloidal Coomassie Blue (BioRad Bio-Safe), and proteins differentially expressed are excised from the gel as 1.5 mm diameter plugs with a OneTouch manual spot picker (The Gel Company). Tryptic digests were performed on the gel pieces and the peptide solutions were evaporated and dried. Mass Spectrometry was performed on a Voyager DE Pro (MALDI-TOF). The filtered peak list was analyzed by peptide mass fingerprinting using Mascot. Certain protein identifications were confirmed on an ABI 4700 MALDI-TOF-TOF instrument at the Pitt Proteomics Core Facility. Detailed description of these proteomics methods could be found in the online supplemental data.

Statistical methods. Fisher's exact test was used to compare seven-day survival rates among mice receiving NTHi treatment at different time points before Spn challenge. Summary statistics for the bacterial counts in lung tissue after Spn were computed within time groups. Analysis of variance (ANOVA) with adjustment for multiple comparisons using Dunnett's test was performed to examine the differences between the mean cell counts of the control group and each of the NTHi treatment groups. Two-way ANOVA of bacterial counts was also performed according to NTHi group (treated vs non-treated) and neutropenic status (neutropenic vs non-neutropenic). All analyses were performed using SAS® (company).

### B. Mouse Model of Pneumococcal Pneumonia

At baseline, the surface epithelium of the intrapulmonary airways of mice and the distal airways of humans shows few or no mucous cells. In response to allergic inflammation (Evans et al., 2004), the airway epithelium changes rapidly to a mucous phenotype such that the majority of cells are filled with electron lucent cytoplasmic granules that stain intensely with Alcian blue and periodic acid Shiff's reagent (AB-PAS). These are striking changes both at the levels of light and electron microscopy, and are generally termed “inflammatory metaplasia.” Similar changes are seen in response to some viruses (e.g., Sendai virus) and to fungal products. Accompanying these structural changes are molecular changes that include upregulation of the mucin gene Muc5ac, the secreted enzyme acidic mammalian chitinase, the calcium-activated chloride channel Gob-5 that may be involved in mucin packaging, and the A3 adenosine receptor that confers exocytic responsiveness to adenosine signaling. Together, these structural and molecular changes appear to augment the apical secretory capacity of the epithelium by increasing the production of secretory products and the molecular machinery for their regulated exocytosis. Presumably, the major physiologic function of these changes is to augment defenses against microbial pathogens, but that hypothesis has received little formal testing, and the selectivity of this response for particular pathogen classes is not well understood. The inventors have generated conditional mutations in mice of key components of the apical exocytic machinery to test the protective and pathologic functions of lumenal secretion in a variety of settings, as described above.

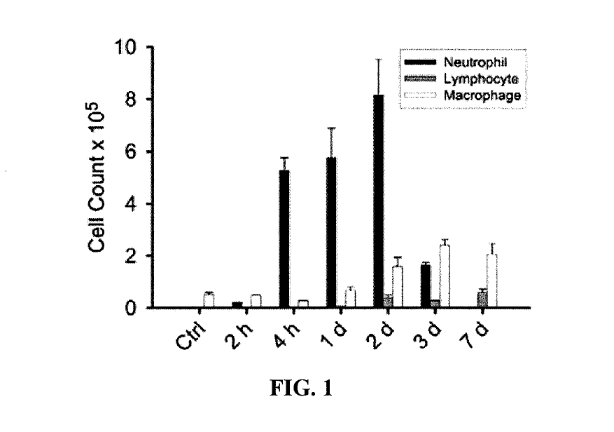

The inventors sought to establish a second mouse model of mucous metaplasia to determine the generalizability of our findings. Bacterial products were initially chosen because of the very different type of inflammatory response they evoke compared to allergic inflammation (a variety of microorganisms are suited for use as a microbial lysate, such as viruses and fungi), and started with NTHi for several reasons. First, NTHi commonly colonizes the airways of patients with COPD and is thought to be a cause of disease exacerbation associated with the acquisition of new strains. It is likely that the lack of a capsule renders the organism less pathogenic, resulting in low grade bronchitis and bronchiolitis rather than invasive pneumonia, but also difficult to clear because of reduced antigenicity compared to encapsulated Haemophilus influenzae. Second, exposure of airway epithelial cells to NTHi products in vitro leads to activation of the Muc5ac promoter in luciferase reporter assays, and signal transduction pathways mediating this response have been extensively analyzed. Based upon this literature, a second model of mucous metaplasia was established by exposing mice to an aerosolized lysate of UV-killed NTHi serotype. The inventors detected no increase in airway mucins either by AB-PAS staining or quantitative real-time RT-PCR, even after repetitive stimulation (not shown). This model was unexpected because neutrophils and neutrophil elastase are thought to induce mucin expression, as is activation of NF-κB, yet there was no induction of mucin despite a robust neutrophilic infiltrate (FIG. 1) and nuclear translocation of NF-κB in epithelial cells (not shown). The inventors recognized the possible advantages of separating the potentially deleterious induction of mucin production from other beneficial aspects of the innate defense mechanisms of the lung epithelium.

To test the functional effects of stimulation of the airway epithelium by an NTHi lysate, the inventors established an aerosol model of pneumococcal pneumonia in BALB/c mice. Streptococcus pneumoniae (Spn) was chosen because it is a highly virulent pathogen in humans and mice, and the most common cause of bacterial pneumonia in humans (ATS/ISDA Guidelines). The pathogen was delivered by aerosol to model the most likely route of delivery of a bioweapon, and because this route results in uniform deposition of a predictable number of organisms in the lungs of a large group of mice, facilitating experimental performance and analysis. Streptococcus pneumoniae (Spn) at concentrations from 1×10⁹ to 1×10¹¹ per ml was aerosolized for 30 min with 5% CO₂ in air to promote deep ventilation.

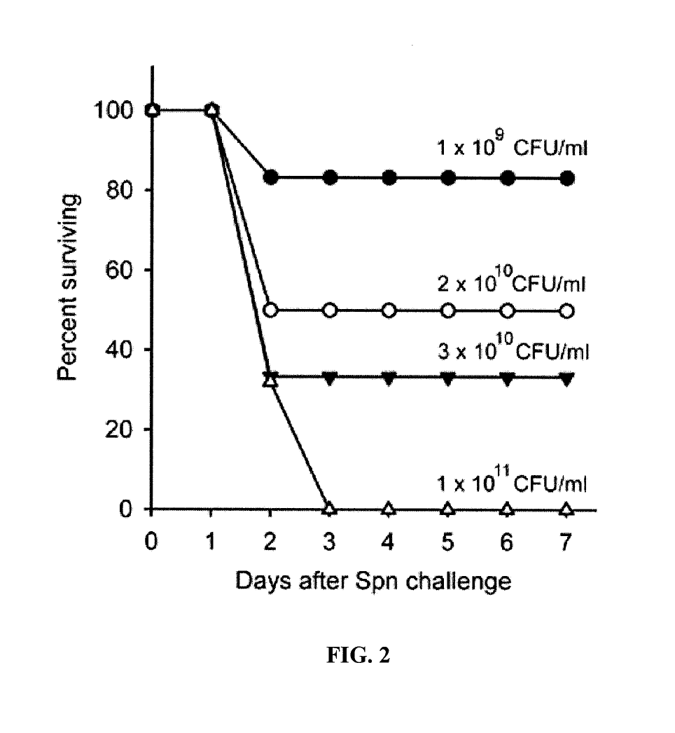

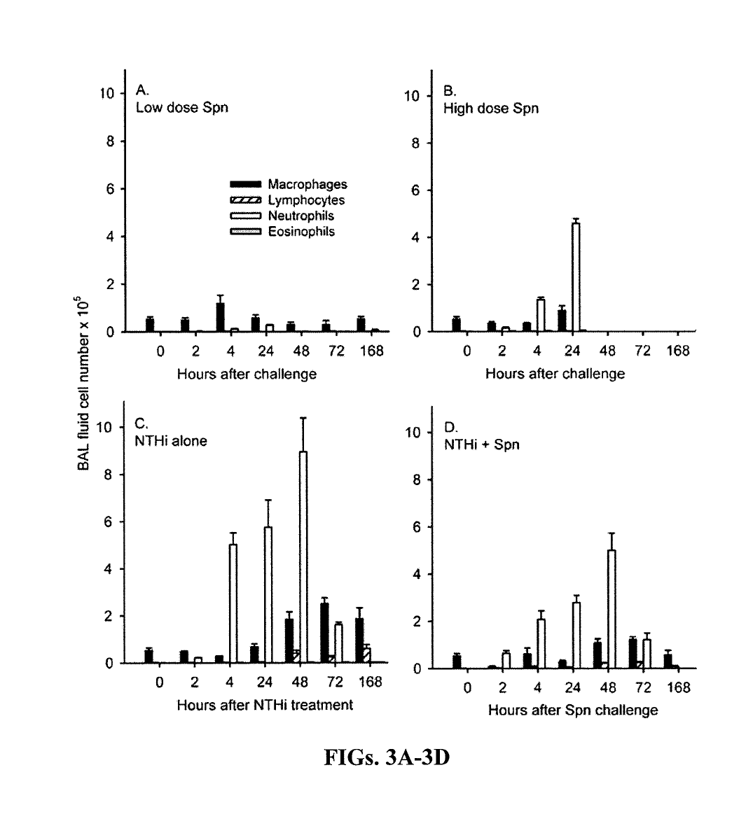

During the first day after aerosol challenge, none of the mice showed adverse effects. However, during the second day, some of the mice began to huddle together, showed ruffled fur and an increased respiratory rate, or were found dead in their cages. There was increasing mortality with exposure to increasing concentrations of Spn, but there was no further mortality after the third day following Spn challenge in any group (FIG. 2). The inflammatory response to Spn was assessed by measuring inflammatory cells in bronchoalveolar lavage (BAL) fluid and examining lung tissue by light microscopy. There was a small increase in neutrophils in lavage fluid during the first day after exposure to Spn at low doses (1×10⁹−2×10⁹ CFU/ml) (FIG. 3A), and no apparent infiltration of lung tissue by inflammatory cells. After exposure to Spn at high doses (4×10¹⁰−1×10¹¹ CFU/ml), neutrophils increased in lavage fluid throughout the first day, and all of the animals were dead by the second day (FIG. 3B). By histochemical analysis, increasing numbers of neutrophils were observed in edematous peribronchial and perivascular connective tissue during the course of the first day, with neutrophils also seen in alveoli at later time points. Bacterial culture of lung and spleen homogenates and of blood immediately after challenge with 4.5×10¹⁰ CFU/ml Spn and again after 48 hr showed 1.5×10⁷ CFU/ml in lung homogenates at time 0 increasing to 1×10⁸ CFU at 48 hr, no organisms in the spleen at time 0 increasing to 2.6×10¹⁰ CFU at 48 hr, and no organisms in blood at time 0 increasing to >10⁸ CFU/ml blood at 48 hr (data not shown). Thus, aerosol challenge of mice with concentrations of Spn less than 2×10¹⁰ CFU/ml resulted in minimal inflammation and falling numbers of viable Spn in the lungs, and low rates of bacteremia or death; in contrast, challenge with concentrations of Spn greater than 4×10¹⁰ CFU/ml resulted in rising neutrophilic inflammation and numbers of viable Spn in the lungs, along with high rates of bacteremia and death during the first 72 hr after challenge.

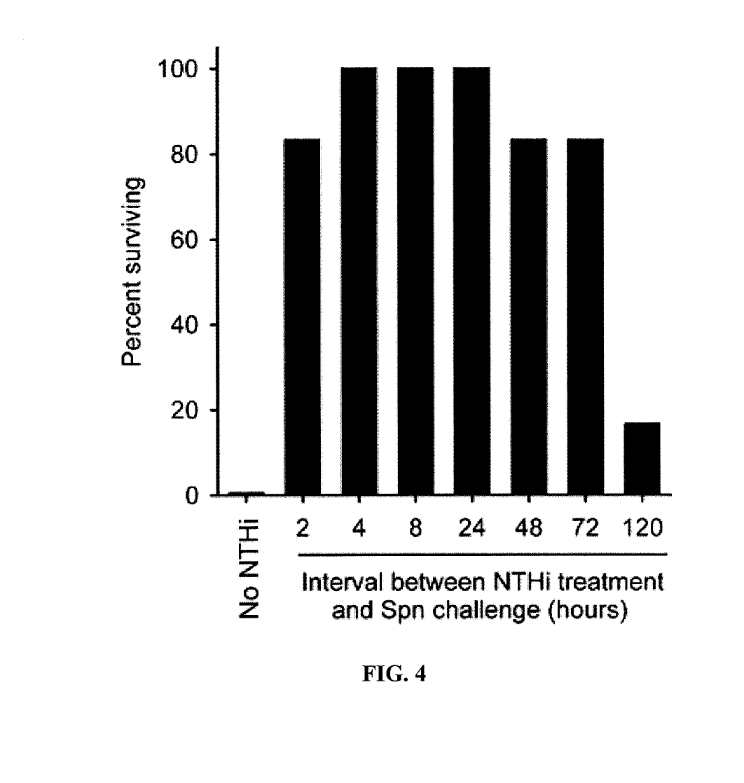

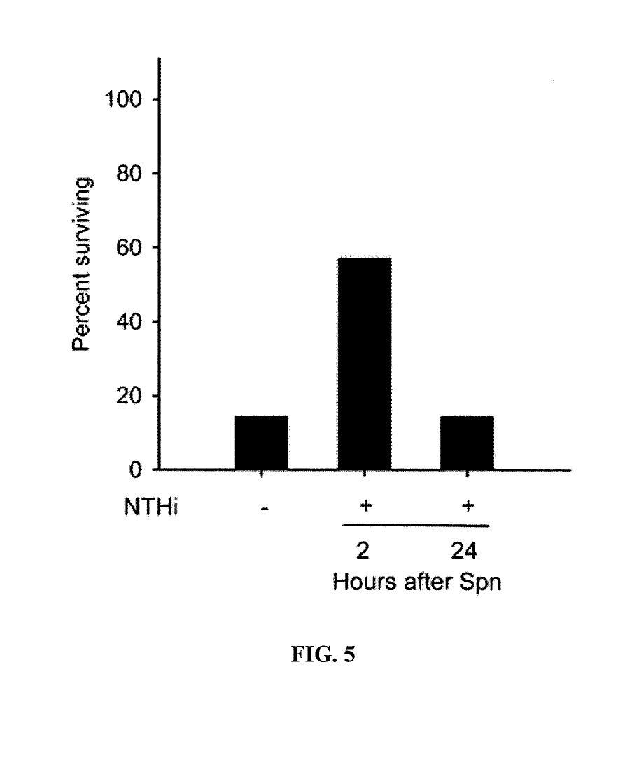

A method of delivering the NTHi lysate was standardized as follows. Bacteria were grown on chocolate agar plates for 24 h at 37° C. in a 5% CO₂ atmosphere, then harvested and incubated for another 16 h in brain-heart infusion broth supplemented with 3.5 μg/ml NAD. The culture was centrifuged at 2500×g for 10 min at 4° C., washed and resuspended in phosphate-buffered saline (PBS). The bacterial suspension was UV-irradiated at 3,000 μjoules/cm² and sonicated for 30 sec. The final protein concentration was adjusted to 2.5 mg/ml in PBS, and aliquots were frozen at −80° C. For protective treatment of mice, a 10 ml sample of the NTHi suspension was thawed and placed in an AeroMist CA-209 compressed gas nebulizer driven by 101/min of room air supplemented with 5% CO₂ to promote maximal ventilation and homogeneous exposure throughout the lungs. A 20 min nebulizing period resulted in the utilization of approximately 6 ml of lysate. The aerosol particles generated were measured using an Andersen cascade impacter and ranged in size from <0.4 to 4.7 μm, with a mass median aerodynamic diameter of 1.49 μm and SD of 1.91 μm. “Preventive treatment” with the NTHi lysate at 2.5 mg/ml for 20 min provided >80% protection from death from Spn challenge for 2-72 h, and 100% protection for 4-24 h (FIG. 4). Even when the bacterial challenge was increased to 5×10¹¹ per ml, there was no death from 4-24 h. Furthermore, there was protection even when the lysate was administered 2 h after the Spn challenge (“preemptive treatment”) (FIG. 5). Since the stimulus is non-cognate to the infectious challenge and host protection develops too rapidly for an adaptive immune response, we conclude that protection results from an inducible innate immune response.

### Example 2

### Stimulation of Lung Innate Immunity Protects Against Lethal Pneumococcal Pneumonia

To stimulate lung innate immunity, mice were exposed to an aerosolized lysate of UV-killed non-typeable Haemophilus influenzae (NTHi). This unencapsulated Gram negative bacterium was chosen because of its relatively distant relation to encapsulated Gram positive Spn to minimize adaptive immune recognition, and because it is a common pathogen in diseases of the airways such as cystic fibrosis, chronic obstructive pulmonary disease, and otitis media. In pilot studies, mice were exposed to increasing concentrations of aerosolized NTHi lysate, with the measurement of neutrophils in BAL fluid used as a marker of the strength of stimulation, and a goal of identifying a stimulus that caused more neutrophilic lung inflammation than high dose Spn. Exposure of mice to an aerosolized NTHi lysate of 2.5 mg/ml for 20 min in an atmosphere of 5% CO₂ resulted in a brisk inflammatory response in the lungs, with the neutrophil number in BAL fluid at 4 hr after NTHi treatment comparable to that at 24 hr in high dose Spn challenge, and maximal neutrophil number at 48 hr (FIG. 4C). A small number of infiltrating lymphocytes and an increase in the number of macrophages was seen at 48 hr, and persisted for 7 days (FIG. 3C). No increase in airway mucin was seen by histochemical staining at any time during the first 7 days (data not shown). Mice treated with the NTHi lysate were then challenged with high dose Spn after varying time intervals. Pretreatment 2 hr before the Spn challenged resulted in an increase in survival from 0% to 83%, and pretreatment from 4 to 24 hr before challenge resulted in 100% survival (FIG. 4). The protective effect of NTHi treatment waned with time such that survival declined to 83% for pretreatment 48 to 72 hr before challenge, and to 17% for pretreatment 5 days before challenge. When mice were pretreated 4 hr before challenge, even the maximal concentration of Spn we were able to deliver by aerosol (5×10¹¹ CFU/ml) caused no mortality (data not shown). In a separate experiment, some protection was also seen when the NTHi treatment was given soon after Spn challenge, with an increase in survival from 14% with no treatment to 57% when treatment was given 2 hr after challenge, but no increase in survival when treatment was given 24 hr after challenge (FIG. 5). Thus, exposure to an aerosolized NTHi lysate provides some protection from lethality when given from 5 days before to 2 hr after challenge with a virulent aerosolized bacterium, and complete protection when given from 24 to 4 hr before challenge.

TABLE 1
Bacterial Counts after Spn Challenge. Lungs Blood Spleen

| | Lungs | | Blood | | Spleen | |
|---|---|---|---|---|---|---|
| | Hr after challenge | | | | | |
| | 0 | 48 | 0 | 48 | 0 | 48 |
| Low dose Spn | 1.5 × 10⁵ | 0 | 0 | 0 | 0 | 0 |
| High dose Spn | 1.5 × 10⁶ | 1 × 10⁸ | 0 | >10⁸ | 0 | 2.6 × 10⁸ |

Low dose Spn challenge was with 2.2 × 10⁹ CFU/ml, and high dose Spn challenge was with 4.5 × 10¹⁰ CFU/ml. Blood and tissue bacterial counts are expressed as CFU/ml of whole blood or tissue homogenates.

### Example 3

### Protection Against Pneumococcal Challenge is Compartment-Specific

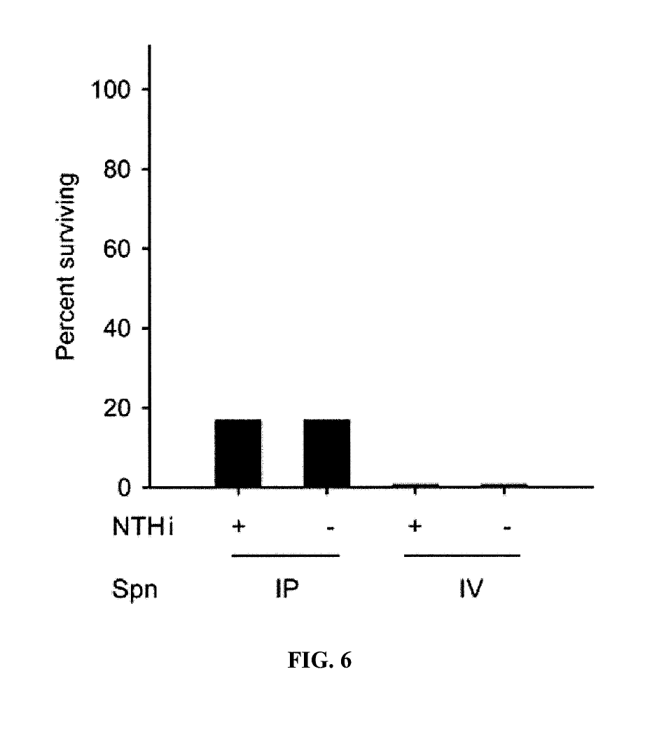

To determine whether stimulation of lung innate immunity results in local or systemic protection against bacterial pathogens, mice pretreated with the aerosolized NTHi lysate were challenged with Spn delivered intravenously or intraperitoneally. Profound neutropenia was induced with either monoclonal antibody RB6-8C5 or cytosine arabinoside. Despite the virtual absence of neutrophils in BAL fluid after NTHi treatment (not shown), mice pretreated with NTHi lysate were protected against pneumococcal challenge (not shown). Next, determination of whether protection is compartment-specific was studied by inoculating Spn intravenously or intraperitoneally. Mortality from 10 CFU introduced by either of these routes was unaffected by pretreatment 4 h earlier with the NTHi lysate aerosol (FIG. 6). The mortality dose-response relationship to intravenous or intraperitoneal injection was determined so that a minimal lethal dose could be used to maximize the chance of identifying a protective systemic effect of the NTHi lysate. These studies revealed that as few as 1×10⁻¹⁰ CFU of Spn were capable of killing mice when delivered systemically by either of these routes (data not shown), so fewer than 10 CFU were used in protection studies. Mice were treated with the NTHi lysate, and then challenged 4 hr later with Spn delivered by intravenous or intraperitoneal injection. Whereas the NTHi lysate had provided complete protection against Spn challenge delivered by aerosol (FIG. 4), it provided little protection against Spn challenge delivered by intravenous or intraperitoneal injection (FIG. 6). The mice challenged with intravenous or intraperitoneal Spn began to die on the second day, and no further mortality occurred in any group after the third day in seven days of observation. Thus, protection against bacterial challenge induced by the aerosolized NTHi lysate is generally localized to the lungs and is not systemic.

### Example 4

### Protection against pneumonia is associated with a Microbicidal Environment in the Lung

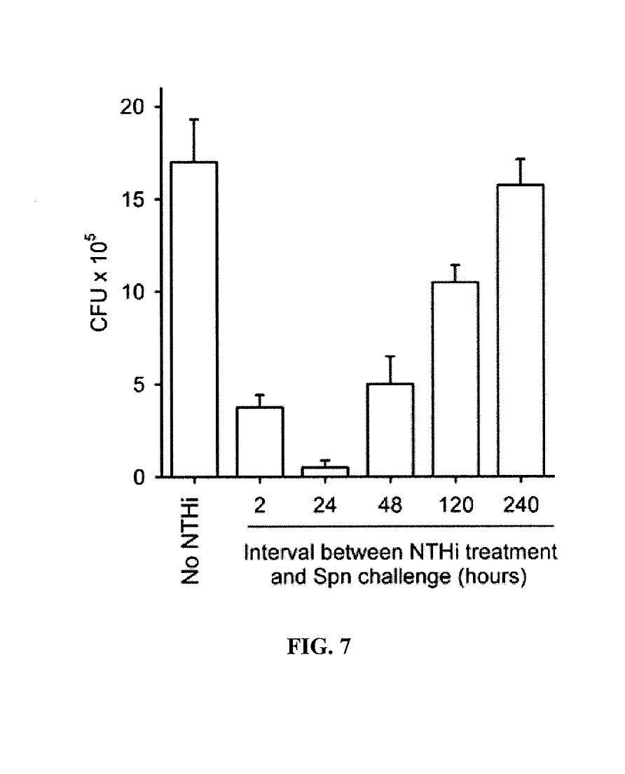

To elucidate the mechanism of protection of the lungs against bacterial challenge, aerosolized NTHi lysate induced bacterial killing was studied. The lungs of mice pretreated with the NTHi lysate at varying times before Spn challenge were excised immediately after exposure to the bacterial aerosol, homogenized, and plated for bacterial culture. The numbers of live bacteria that could be cultured from the lungs correlated inversely with protection against lethal pneumonia, such that 1.7×10⁶ CFU were present in the lungs of naïve mice, but only 1×10⁵ CFU in the lungs of fully protected mice 24 hr after NTHi pretreatment (FIG. 7). Intermediate numbers of viable bacteria were present in the lungs of mice with intermediate levels of protection during the rising and falling limbs of the survival curve (FIG. 4 and FIG. 7). From these data, it is inferred that one mechanism of protection against lethality induced by the NTHi lysate is local killing of Spn before they cross lung mucosal barriers, since access of even small numbers of Spn to the vascular space or internal compartments of mice rapidly leads to death of the host (FIG. 7). Therefore, the mechanism of bacterial killing was assessed.

### Example 5

### Protection Against Lethality does not Depend Upon Inflammatory Cell Recruitment, Though the Lung Microbicidal Environment Depends Partially

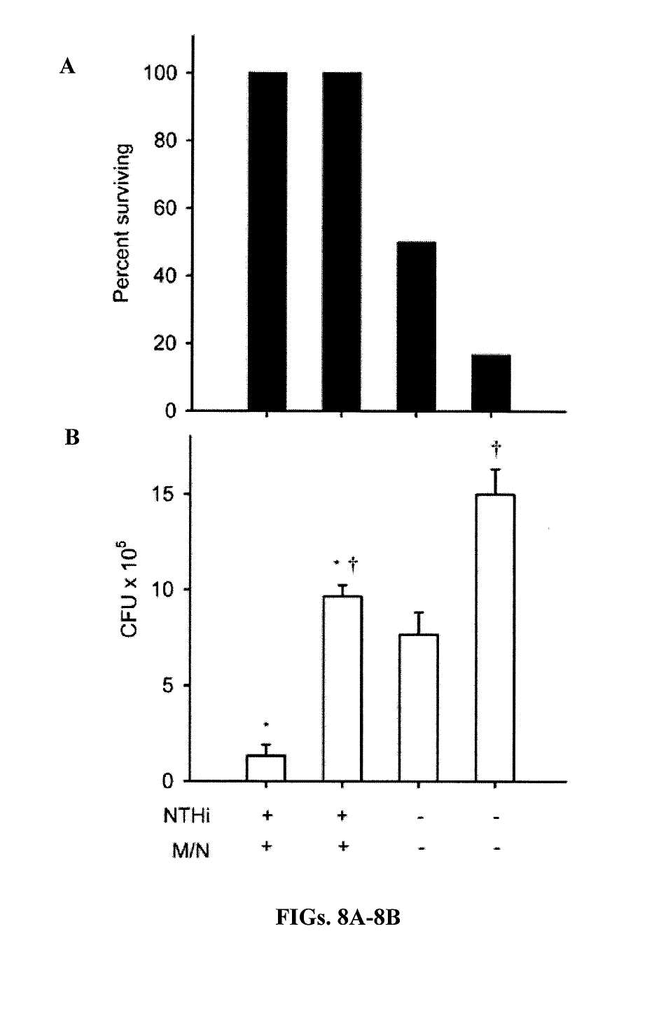

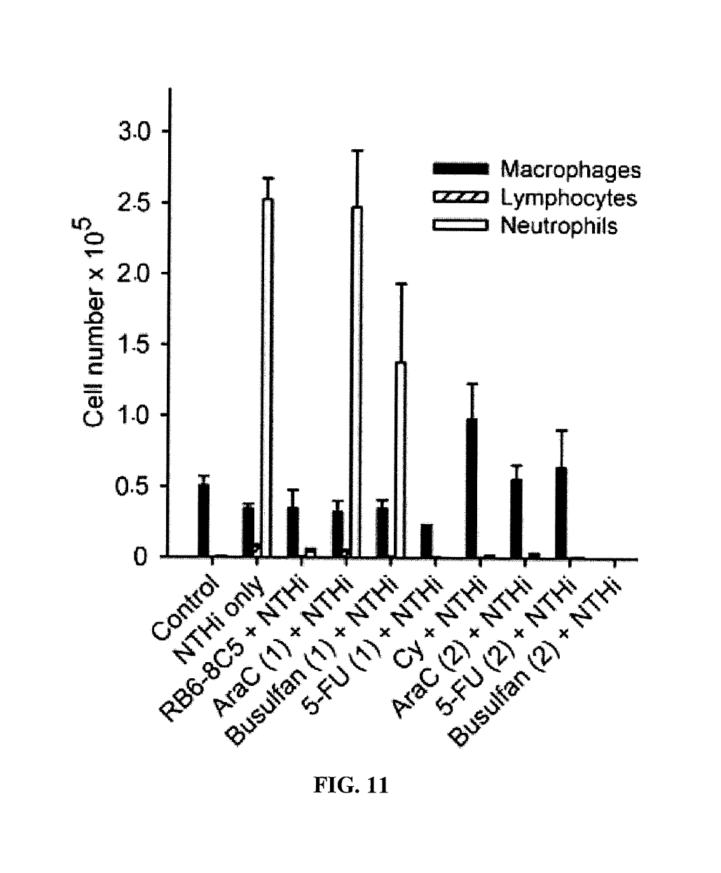

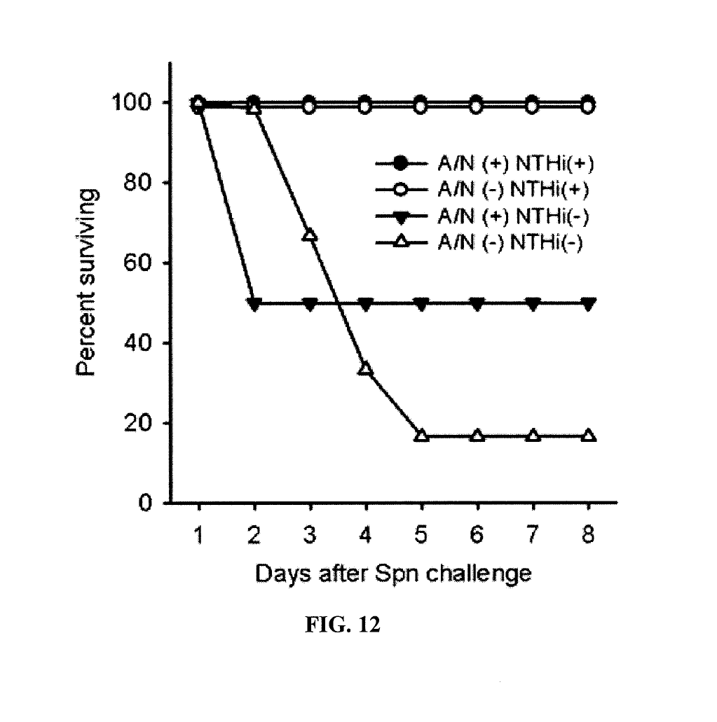

Initially the aerosolized NTHi lysate stimulus had been titrated to neutrophil recruitment to the lungs, and the time course of neutrophil influx and resolution roughly parallels that of protection (FIG. 3 and FIG. 4). Therefore, neutrophil recruitment to the lungs was assessed as a requirement for protection by the NTHi lysate against Spn pneumonia by preventing neutrophil influx. In pilot studies, several protocols to prevent neutrophil influx in response to the aerosolized NTHi lysate were tested, using antibody directed against neutrophils to induce neutrophil lysis, and alkylating agents or nucleoside analogs to suppress hematopoiesis. Intravenous rat monoclonal antibody against mouse neutrophils reduced BAL neutrophil numbers 24 hr after NTHi treatment by 96% from 2.5×10⁵ to 0.1×10⁵ (FIG. 11), and was used in most subsequent experiments. In addition, the numbers of alveolar macrophages were reduced in BAL fluid by 70% using aerosolized liposomal clodronate to assess their participation in protection. All the mice pretreated with NTHi survived challenge with intermediate dose Spn whether or not they were depleted of alveolar macrophages and neutrophils (M/N), compared to 50% lethality among M/N sufficient mice not treated with NTHi, and 83% lethality among M/N depleted mice not treated with NTHi (FIG. 8, top). Death among M/N depleted mice not treated with NTHi continued to occur after 3 days (FIG. 12), different from all experiments with M/N sufficient mice in which mice that survived the first 3 days did not subsequently die. Bacterial counts in the lungs of either M/N depleted but NTHi treated mice, or M/N sufficient but NTHi untreated mice, were intermediate between the high bacterial counts in M/N depleted and NTHi untreated mice, and the low counts in M/N sufficient and NTHi treated mice (FIG. 8, bottom).

These results suggest that protection from lethality by treatment with the aerosolized NTHi lysate does not depend upon neutrophil recruitment or alveolar macrophages, but that rapid bacterial killing in the lungs of treated mice and late mortality in untreated mice depends partially upon neutrophils and macrophages. Since it was possible that the anti-neutrophil antibodies and clodronate induced protection against lethality through lysis-induced inflammatory mechanisms despite a severe reduction in neutrophil number and moderate reduction in macrophage number, the inventors also tested the role of neutrophil recruitment in protection by suppressing hematopoiesis with the nucleoside analog cytosine arabinoside. Using a high-dose, short-term regimen of intraperitoneal cytosine arabinose that prevented any detectable rise in BAL fluid neutrophils in response to treatment with the NTHi lysate (Table 1 and FIG. 11), but without clodronate to kill alveolar macrophages, similar results were obtained to those with the antineutrophil antibody and clodronate, with independence of the NTHi lysate from neutrophils in protection against lethality during the first 72 hr, and partial dependence upon neutrophils in bacterial killing within the lung (data not shown). These mice all died from bone marrow failure on the fourth and fifth days after Spn challenge, similar to mice treated with cytosine arabinoside without Spn challenge (FIG. 2). Thus, protection by the NTHi lysate from lethal Spn aerosol challenge does not depend upon neutrophil recruitment to the lungs, but rapid bacterial killing appears to depend partially upon alveolar macrophages.

TABLE 2
Neutrophil Depletion Regimens. Mice were pretreated with various
regimens to reduce neutrophil recruitment to the lungs. The timing of
doses is listed as the number of days prior to NTHi treatment, with
day 0 being the day of NTHi treatment, day −1 being one day prior to
NTHi treatment, etc.

| Agent | Dose | Timing | Route |
|---|---|---|---|
| RB6-8C5 | 50 μg | Days −1, 0 | IV |
| Ara-C (1) | 300 mg/kg | Days −8, −5, −2, −1 | IP |
| Ara-C (2) | 600 mg/kg | Days −5, −2, −1, 0 | IP |
| Busulfan (1) | 125 mg/kg | Days −8, −5, −2, −1 | IP |
| Busulfan (2) | 125 mg/kg | Days −11, −8, −6, −4, −1 | IP |
| 5-FU (1) | 150 mg/kg | Days −8, −5, −2, −1 | IP |
| 5-FU (2) | 150 mg/kg | Days −8, −3 | IP |
| Cyclophosphamide | 200 mg/kg | Days −5, −2, −1 | IP |

* Abbreviations are as follows:
RB6-8C5 - rat monoclonal antibody against mouse neutrophils;
Ara-C—cytosine arabinoside;
5-FU—5-fluorouracil;
IV—intravenous;
IP—intraperitoneal.

### Example 6

### Protection against pneumonia is associated with secretion into the Lung Lining Fluid of Multiple Antimicrobial Polypeptides

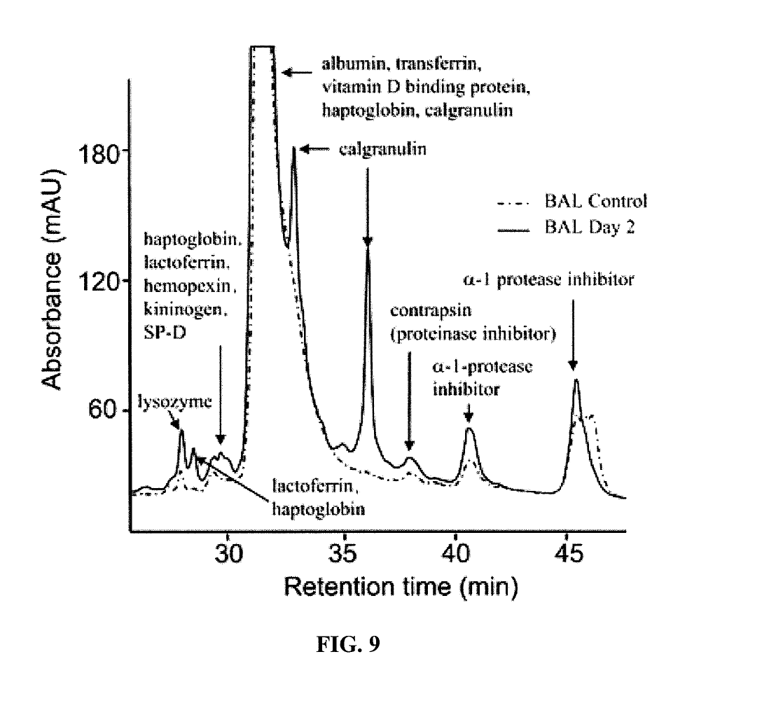

Since bacterial killing in lungs stimulated with the aerosolized NTHi lysate was rapid and only partially dependent on inflammatory cells, it was suspected that the lysate might stimulate the production and secretion of antimicrobial polypeptides from lung parenchymal cells. Proteomic analysis of bronchoalveolar lavage (BAL) fluid was performed to identify potential antimicrobial proteins that might mediate host protection and bacterial killing. BAL fluids obtained from mice 48 h after exposure to NTHi lysate were compared to those from unexposed mice by reversed phase HPLC coupled with LC-MS/MS (FIG. 9), differential two-dimensional gel electrophoresis (DIGE) using Cy3 and Cy5 labeled proteins in single gels (FIGS. 11A-11B), and quantitative isobaric stable isotope tag (iTRAQ) labeling with LC-MS/MS (FIGS. 12A-12C).

By mass spectrometry, these differential peaks were found to include multiple antimicrobial polypeptides including lysozyme, lactoferrin, haptoglobin, calgranulin, and surfactant apoprotein D. Similarly, differential gel electrophoresis analysis and isobaric stable isotope labeling (FIG. 12) identified multiple increased antimicrobial polypeptides in the treated samples. Two dimensional difference gel electrophoresis analysis of proteins present in bronchoalveolar lavage fluid after treatment with NTHi lysate included collecting and precipitating BAL fluid supernatants then dissolving the precipitant in denaturing lysis buffer containing 7 M urea, 2 M thiourea, 4% CHAPS, and 1% Triton X-100. One hundred μg of each of two protein samples from the lungs of mice that were untreated (BAL control) or pretreated 48 hr previously with NTHi lysate (BAL day 2) were labeled on lysine residues with Cy3 or Cy5 fluorescent dyes, then electrofocused in pH 3-10 isoelectric focusing strips, followed by electrophoresis in 10% acrylamide gels. Cy3 and Cy5 images were acquired and stacked, and a two-frame movie evaluated for differences in spot intensity, with at least two independent comparisons performed to identify repeatable differences. Gels were then stained with colloidal Coomassie Blue, excised from the gel as 1.5 mm diameter plugs, digested with trypsin, analyzed by MALDI-TOF MS, and identified by database searching. Proteins identified as elevated in the treated mice include: (1) polymeric Ig receptor, (2) lymphocyte cytosolic protein 1, (3) haptoglobin, (4) arghdia, (5) serpin bla, (6) complement 3c, (7) leukotriene E4 hydrolase, (8) enolase 1, (9) surfactant apoprotein D, (10) WD repeat domain protein 1, (11) transketolase, (12) glucose phosphate isomerase 1, (13) chitinase 3-like protein 1, (14) lipocalin, (15) lactoferrin.

Some increased polypeptides were identified by two of the three techniques, and a few by all three techniques (Table 3). Altogether, increased amounts of various polypeptides with possible antimicrobial activity were identified by the three techniques. Some of these polypeptides, such as lysozyme, chitinase-3, and surfactant apoprotein D, have been reported to be expressed by lung epithelial cells. Others such as lymphocyte cytosolic protein 1 have been reported to be expressed by leukocytes, and some such as calgranulin B have been reported to be expressed both by epithelial cells and leukocytes. Thus, protection from lethality from pneumococcal pneumonia and increased local antimicrobial activity induced by the aerosolized NTHi lysate are associated with increased amounts of antimicrobial polypeptides in lung lining fluid. The inventors conclude that protection by NTHi lysate results from localized upregulation of innate immune defense mechanisms that result in rapid killing of bacteria introduced through the airways.

TABLE 3
Proteomic Analysis of NTHi-Treated BAL Fluid.

| | Proteomic Technique | | |
|---|---|---|---|
| Identified Protein (GenBank Accession #) | HPLC | iTRAQ | DIGE |
| Pulmonary surfactant-associated protein D | • | • | • |
| (NP_033186) | | | |
| Haptoglobin-2 (NP_059066) | • | • | • |
| Calgranulin B (P31725) | • | • | |
| Kininogen (AAH18158) | • | • | |
| Chitinase-3-like protein 1 (NP_034022) | | • | • |
| Complement C3 (AAH43338) | | • | • |
| Transferrin (NP_598738) | • | | • |
| Lactoferrin (NP_032548) | • | | • |
| Lysozyme (NP_059068) | • | | |
| Alpha-1-protease inhibitor (P22599) | • | | |
| Hemopexin (NP_059067) | • | | |
| Vitamin D binding protein (AAA37669) | • | | |
| Hemoglobin alpha chain (P01942) | | • | |
| Hemoblogin beta chain (P02088) | | • | |
| Alpha-1-acid glycoprotein 1 (NP_032794) | | • | |
| Inter-alpha-trypsin inhibitor heavy chain H4 | | • | |
| (NP_061216) | | | |
| Transketolase (NP_033414) | | | • |
| Serpin 1 A protein (NP_079705) | | | • |
| Glucose phosphate isomerase (NP_032181) | | | • |
| Rho GDI alpha (NP_598557) | | | • |
| Indolethylamine N-methyltransferase | | | • |
| (NP_033375) | | | |
| Aldehyde dehydrogenase 1A1 (AAH44729) | | | • |
| Polymeric immunoglobulin receptor | | | • |
| (NP_035212) | | | |
| Leukotriene A4 hydrolase (NP_032543) | | | • |
| Gamma actin (CAA31455) | | | • |
| Lymphocyte cytosolic protein 1 (AAH22943) | | | • |
| Lipocalin 2 (NP_032517) | | | • |

### Example 7

### Repetitive Exposure to Bacterial Products

The NTHi aerosol model was used to assess the possible role of repetitive exposure to bacterial products in progression of the structural changes seen in patients with COPD. Only 15-20% of smokers develop COPD, indicating differences among individuals in genetic susceptibility or exposure to environmental factors besides smoke. Further, COPD patients show a progressive decline in lung function even after smoking cessation. One susceptibility factor that has been suggested based upon cross-sectional and longitudinal studies is repetitive or chronic bacterial colonization of smoke-damaged airways, particularly by NTHi. Data from the inventors studies testing this hypothesis can serve as long term toxicity data.

Mice were exposed weekly for 50 weeks to the NTHi lysate aerosol at 2.5 mg/ml for 20 min, as for the acute experiments. No obvious gross pathology was observed, such as premature death, weight loss or behavioral changes. The extent of neutrophilic inflammation measured in BAL fluid one day after NTHi exposure declined progressively at weeks 8, 25, and 50 (FIG. 11). After each NTHi exposure, BAL neutrophilia resolved over the course of one week (data not shown), similar to the first exposure. One week after 8 weekly exposures, a mononuclear cell infiltration was observed histopathologically surrounding airway walls though only very few inflammatory cells were observed at any time point in the alveoli (not shown). Immunohistochemical staining of the airway wall inflammatory cells revealed abundant CD68+ macrophages, CD8+ cytolytic T cells, and CD20+ B cells, with rare CD4+ helper T cells (not shown). Analysis of cytokines and chemokines in BAL fluid after 8 weekly exposures showed marked elevation at 4 h of inflammatory (e.g., TNF-α, IL-6) and Th1 (e.g., IFN-γ) cytokines, as well as the neutrophil chemokine KC. The neutrophilic, CD8+, Th1+ inflammatory response in our mouse model is characteristic of inflammation in COPD patients, though no structural changes characteristic of COPD were observed, such as mucous metaplasia, airway wall fibrosis, or alveolar enlargement. However, after 25 weekly exposures, in addition to the inflammatory cell infiltrate, airway wall fibrosis was apparent by Masson's trichrome staining, and after 50 weekly exposures, alveolar enlargement as seen as well. In summary, repetitive weekly exposure to the aerosolized NTHi lysate results in infiltration of the airway wall with chronic immune cells but no apparent structural changes by 8 weeks, and airway wall fibrosis by 25 weeks.

TABLE 4
Cytokine levels post administration of a lysate.

| | Time | | | | | | | |
|---|---|---|---|---|---|---|---|---|
| Cytokine | Ctrl | 4 h | 1 d | 2 d | 3 d | 7 d | 14 d | 28 d |
| TNF-α | 4.99 | 14545.80 | 20.94 | 6.85 | 5.94 | 5.86 | 4.41 | 3.55 |
| IFN-γ | 21.93 | 541.45 | 240.85 | 27.59 | 35.52 | 54.30 | 80.93 | 51.88 |
| KC | 7.09 | 111.89 | 3.82 | 12.54 | 15.88 | 26.26 | 21.02 | 13.98 |
| IL-1β | 7.06 | 41.42 | 2.51 | 11.26 | 12.13 | 8.77 | 14.09 | 10.83 |
| IL-4 | 0.47 | 10.19 | 0.50 | 1.06 | 1.77 | 1.40 | 1.11 | 1.23 |
| IL-6 | 9.08 | 59873.75 | 318.32 | 18.83 | 28.69 | 25.20 | 18.40 | 19.30 |
| IL-10 | 0.00 | 43.86 | 0.00 | 0.00 | 0.30 | 0.00 | 0.00 | 0.00 |
| IL-12 | 2.44 | 49.45 | 0.54 | 1.44 | 0.90 | 2.27 | 1.37 | 1.06 |
| IL-13 | 5.23 | 83.26 | 8.51 | 10.27 | 10.02 | 14.06 | 25.09 | 12.34 |
| Eotaxin | 74.06 | 1374.78 | 142.85 | 174.47 | 373.39 | 217.33 | 166.71 | 183.00 |

### Example 8

### Range of Protection of Aerosolized NTHi Lysate Against Various Pathogens

It is contemplated that aerosolized NTHi lysate will provide broad protection against a wide array of respiratory pathogens because the signaling molecules released locally (e.g., IFN-γ, TNF-α, eicosanoids) are known to stimulate defense against multiple pathogen classes, and the upregulated polypeptides identified in the lung lining fluid (e.g., lysozyme, lactoferrin, cathelicidins) have broad antimicrobial specificity. The inventors assessed the exemplary bacteria Francisella novicida and Francisella tularensis because they are highly pathogenic, are intracellular pathogens, and the latter is a Class A bioterror agent. The inventors contemplate assessing other organism, with the results described being exemplary of and applicable to a number of pathogens.

Streptococcus pneumoniae (pneumococcus). The inventors have extensively analyzed pneumonia with this gram positive bacterial pathogen delivered by aerosol, wherein aerosolized NTHi lysate provides complete protection against the highest numbers of organisms that are able to deliver by aerosol (5×10¹¹/ml). This provides proof of the efficacy of aerosolized microbial lysate, exemplified by NTHi lysate, against the most likely method of delivery of bioweapon pathogens. However, the principal mechanism of delivery of pneumocci to the lungs in the civilian setting is thought to be by aspiration of oropharyngeal contents. It is possible that localized delivery of high numbers of organisms to the lower respiratory tract by aspiration could overwhelm lung defenses stimulated by aerosolized NTHi lysate. Therefore, the inventors will assess the protection against increasing numbers of organisms delivered by nasal installation of 50 μl into sedated mice. As in the aerosol model described above, a minimal number of organisms that result in 100% mortality will be determined. Using this number of organisms the inventors will assess protection by aerosolized NTHi lysate against that number and a 100-fold higher number of pneumococci. It is contemplated that the microbial lysates will provide some measure of protection, if not complete protection from bacterial challenge. If aerosolized NTHi lysate does not provide complete protection against lethality from the high-concentration pathogen challenge, serial three-fold higher numbers of organisms from 1 to 81 times the minimal lethal number will be used to find the maximal number against which aerosolized NTHi lysate provides full protection.

Pseudomonas aeruginosa. This organism will be tested both by aerosol to further assess the efficacy of aerosolized NTHi lysate as a biodefense model in general, and by nasal installation to test its efficacy against this specific pathogen in a model that more closely mimics human infection in immunocompromised civilians. The aerosolized model is well established in the cystic fibrosis literature. Colleagues of the inventors have extensive experience with the nasal instillation model, and are familiar with the strain-specific requirements to induce mortality.

Klebsiella pneumoniae. The inventors will use this second gram negative organism to test the efficacy of aerosolized NTHi lysate against both aerosol and aspiration challenges because the low pathogenicity of Pseudomonas precludes generalizing from results with that organism alone. Klebsiella has been used by intraperitoneal injection to test the role of mast cell degranulation in protection against infection using Syt-II mutant mice (unpublished results). For the pneumonia models, the inventors will test serial dilutions of Klebsiella to find concentrations that result in 100% mortality, then measure preventive and preemptive efficacy of aerosolized NTHi lysate at various time points.

Aspergillus fumigatus. This ubiquitous organism is delivered to the lower respiratory tract as aerosolized conidia, and normal subjects are exposed daily. It generally does not cause disease in immunocompetent hosts, though it may contribute to allergic airway inflammation in asthmatics, can lead to more severe localized airway inflammation when hyphal forms grow in impacted mucus of allergic subjects suffering from allergic bronchopulmonary aspergillosis (ABPA), and can colonize and cause inflammation in the walls of preexisting anatomical cavities in the lungs (aspergilloma). In immunocompromised subjects, Aspergillus is a serious opportunistic pathogen that causes invasive disease with a high mortality rate. Mouse models of Aspergillus pneumonia are available and inhalation delivery has been optimized for reproducible inhalational delivery of conidia using a device similar to the nebulizer the inventors use for delivery of aerosolized NTHi lysate. The specific requirements for immunosuppression and suppression of bacterial co-infection have also been defined.

Parainfluenza (Sendai) and influenza viruses. In normal human hosts, parainfluenza generally causes only a mild upper respiratory infection, though in asthmatics it can contribute to worsening of airway inflammation, and in immunocompromised hosts such as those who have undergone hematopoietic stem cell transplantation it can cause life-threatening bronchiolitis or pneumonia. In mice at low doses, it causes persistent mucous metaplasia of the airway epithelium that resembles one aspect of human asthma, and at high doses it can cause lethal pneumonia. Influenza causes respiratory illness in humans that ranges from mild to severe even in normal hosts.

In brief, parainfluenza virus type 1 (Sendai virus) and influenza virus A/PR/8/34 (H1N1) are grown in monolayers of rhesus monkey kidney cells. After one week, cultures are frozen and thawed to disrupt cells, the fluid cleared by low speed centrifugation, and supernatants titered to determine the concentration that causes infection of 50% of cell monolayers (TCID₅₀) and stored in aliquots at −70° C. Anesthetized mice are infected by nasal instillation of 5×10⁴ TCID₅₀ of either virus in a 50 μl volume. Infected mice are kept in laminar flow hoods, and four days after infection, are euthanized. A lobe from each animal is frozen at −70° C. for measurement of viral content by real time RT-PCR of viral RNA and by viral titer expressed as TCID50 per gram lung wet weight. The remaining lung is lavaged for inflammatory cell counts.

To determine the effect of treatment with aerosolized NTHi lysate on virus induced mortality, mice with be infected with serial two-fold dilutions of virus stock containing from 40 to 10,240 TCID₅₀ of virus, then observed for two weeks. Mice begin to die at the end of the first week, and all mice that will die have done so by the end of the second week. Both influenza and parainfluenza cause a dose related mortality that can be quantified and expressed as an LD₅₀ (the amount of virus that causes 50% mortality). A change in the LD₅₀ with treatment with NTHi lysate will indicate an effect on virus induced mortality.

Francisella species. Francisella novicida is used in a mouse model of pneumonic tularemia. In brief, strain U 112 is cultured overnight, pelleted, and resuspended in PBS at 1×10⁹ CFU/ml. As many as 24 mice are placed in restraining tubes that are then mounted on an In-Tox chamber (Sputnik), and 5 ml of the bacterial suspension placed in an in-line Uni-Heart nebulizer with a flow rate of 15/min for 10 min that delivers a bacterial aerosol through the chamber. At various times after exposure, animals are euthanized and their lungs, liver, kidney and spleen harvested to determine bacterial counts and dissemination of infection. This infection is uniformly lethal in wild type mice, even with inocula of fewer than 10 bacteria, and triggers almost no host response. Therefore, the inventors expect that innate immune stimulation with aerosolized NTHi lysate will markedly change the course of disease.

Bacillus anthracis. This is a Class A bioterror agent that causes disease primarily by production of three virulence factors—edema toxin, lethal toxin, and a weakly antigenic poly-D-glutamic acid capsule.

The inventors contemplate protection by aerosolized NTHi lysate against all the extracellular bacterial pathogens because of the high level of protection it provides against a virulent pneumococcal strain, and relatively non-pathogen-specific mechanisms of action of innate defenses. It is possible that the dose-response curve for protection against bacterial delivery by aspiration will differ from that against delivery by aerosol. Protection by aerosolized NTHi lysate against intracellular bacterial pathogens, Aspergillus and viruses is less predictable. Based upon the rapid bacterial killing seen in the pneumococcal model, the non-pathogen-specific actions of innate defenses, and the high levels of IFN-γ and TNF-α, the inventors contemplate efficacy against these pathogens as well.

TABLE 5
Protection by bacterial lysates given by aerosolization 24 hours before a
challenge by lethal dose of Streptococcus pneumoniae.

| | Gram | Protection, |
|---|---|---|
| Lysate (at 2.5 mg/ml) | pos/neg | degree of |
| NONTYPEABLE | − | 100% |
| HAEMOPHILUS | | |
| INFLUENZAE | | |
| PSEUDOMONAS | − | 100% |
| AERUGINOSA | | |
| ESCHERICHIA COLI | − | 100% |
| STAPHYLOCCOCUS AUREUS | + | 100% |
| STREPTOCOCCUS | + | 50% |
| PNEUMONIAE | | |

### Example 9

### Dose-Response and Time-Response Relationships of Aerosolized NTHi Lysate

The inventors have found that aerosolized NTHi lysate delivered at a protein concentration of 0.25 mg/ml provides only modest protection against pneumococcal pneumonia (not shown), but at 2.5 mg/ml provides a high level of protection (FIG. 5). To use the minimal effective dose in order to minimize side effects, it will be necessary to more precisely determine the dose-response relationship. This will also help precisely compare the relative protective efficacy of aerosolized NTHi lysate against different pathogens. The inventors will determine the dose-response relationship for one pathogen in each class (intracellular bacterial pathogen, gram positive and gram negative extracellular bacteria, fungi and viruses), assuming that the innate antimicrobial mechanisms stimulated by aerosolized NTHi lysate have similar efficacy within a class. The inventors will also test how the dose-response relationship for preemption compares to that for prevention, since they can be expected to be different.

The pathogens that will be used are Francisella novicida, Streptococcus pneumoniae, Klebsiella pneumoniae, Aspergillus fumigatus, and Sendai virus. Five groups of six mice each will be tested at doubling concentrations of aerosolized NTHi lysate from 0.25-4.0 mg/ml for protection against twice the minimal dose of pathogen associated with 100% mortality. This experiment will be performed at two time points—one day prior to pathogen administration to test the preventive dose-response relationship, and two hours after pathogen administration to test the preemptive dose-response relationship. In addition, groups of mice will be tested at the minimal fully protective preventive dose for each pathogen at two hours prior to pathogen challenge to assess the rising limb of the time-response curve, and at three days prior to challenge to test the falling limb. This last study will establish whether a single preventive dose provides sustained protection from three days to two hours prior to pathogen exposure. The inventors are also studying whether twice the concentration given for half the time (i.e., ten min) shows equal efficacy against Streptococcus pneumoniae, which would allow more efficient delivery under emergency conditions.

Based upon preliminary studies with pneumococcal pneumonia, the inventors contemplate finding minimal protection at the lowest dose and full protection at the highest dose, and expect to find partial protection at intermediate doses. The inventors anticipate similar sigmoidal dose-response curves for all four pathogens. For the time-response curves, it is possible that a preventive dose that is fully protective given one day prior to pathogen challenge will be only partially protective given three days prior to challenge. In that case, the inventors will repeat the study at the next higher dose level to try to find a single dose that provides protection from two hours to three days for ease of dosing in the clinical setting. The inventors expect the preemptive dose-response curve to be shifted to the right compared to the preventive curve, and think it would be reasonable to recommend a second (higher) dose of aerosolized NTHi lysate for preemptive treatment of patients already exposed to a pathogen.

### Example 10

### Efficacy and Toxicity Dose-Response Curves of Aerosolized NTHi Lysate

Activation of the epithelium to produce apically secreted antimicrobial polypeptides appears to be the major protective mechanism of aerosolized NTHi lysate, although other antimicrobial molecules such as reactive oxidant species may also participate, and the precise protective roles of individual molecular species have not yet been formally tested. In addition to increasing the production and apical secretion of antimicrobial molecules, the epithelium releases cytokines and chemokines that cause systemic inflammation and recruit leukocytes to the lungs. While leukocyte recruitment can be an important mechanism to contain infection not controlled by local innate immune mechanisms, it is associated with symptoms of systemic inflammation such as lassitude and fever that may limit the utility of aerosolized NTHi lysate in some settings such as during active military duty, and eventually leads to an adaptive immune response that limits the safety of repetitive dosing. Remarkably, glucocorticosteroids can suppress the systemic release of cytokines and chemokines without suppressing local innate defenses. The protective role of steroids in acute stress responses is well known, and some innate defenses of the lungs are actually increased by steroids. Considerable experience during the past twenty years with steroids delivered topically to the lungs by aerosolization demonstrates benefit in the treatment of diseases such as asthma, allergic rhinosinusitis, allergic bronchopulmonary aspergillosis, and COPD without increased infection. To optimize the cost-benefit ratio of treatment with aerosolized NTHi lysate, the inventors contemplate compositions with and without added steroids.

To determine the improvement by beclomethasone in side effects from aerosolized NTHi lysate, the inventors measure the dose-response relationship of body temperature, systemic leukocyte count, BAL leukocyte count, systemic and BAL cytokine and chemokine levels (TNF-α, IFN-γ, IL-8), and hepatic acute phase reactants (LBP-LPS binding proteins, haptoglobin, and serum amyloid protein) to doubling doses of beclothemasone from 0.25 mg/2.5 ml (the FDA-approved concentration for the treatment of asthma and COPD) to 2 mg/2.5 ml added to the optimal preventive dose of aerosolized NTHi lysate. The inventors will then determine the effect of added beclomethasone on protective efficacy of aerosolized NTHi lysate by measuring bacterial counts in lung homogenates taken four hours after challenge with 10-11 aerosolized pneumococci. The inventors will also measure the effect on Sendai virus infection by RT-PCR and infectious titers four days after challenge to be certain that the antiviral protective effect is not impaired by a reduction in local cytokine levels.

The inventors will also perform an acute dose-escalation toxicity study. The inventors have observed no acute toxicity in numerous experiments at a dose (2.5 mg/ml) that is highly protective for a virulent pathogen. Further, the inventors have tested the toxicity of chronic dosing at this level and not found dose-limiting toxicity until 25 exposures. Nonetheless, it will be helpful for the dog and human toxicity studies to know the level at which acute serious adverse events occur. The inventors will use serial doubling concentrations starting at 2.5 mg/ml and increasing until the nebulizer ceases to function effectively because of viscosity of the suspension, which is expected to occur around 20-40 mg/ml.

The inventors will also assess possible changes in lung mechanics (physiology) following treatment with aerosolized NTHi lysate. Allergic airway inflammation leads to bronchospasm and bronchial hyperreactivity, mediated predominantly by the effects of IL-13 on airway smooth muscle. The levels of IL-13 and other Th2 cytokines are low following exposure to aerosolized NTHi lysate, but it is nonetheless possible that other inflammatory mediators could induce bronchial hyperreactivity. This will be measured as a change in baseline dynamic lung compliance 15 min, 4 hours, and 24 hours after treatment with aerosolized NTHi lysate, and in response to increasing doses of intravenous methacholine. It is also possible that penetration of aerosolized NTHi lysate to the distal airspaces will result in some alveolar edema, although no accumulation of neutrophils or edema fluid within alveoli was seen histopathologically in mice. This will be measured as a change in quasi-static lung compliance at the same time points.

The inventors do not expect to find impairment by added beclomethasone of the protective response to aerosolized NTHi lysate, at least at low to moderate doses of beclomethasone. The inventors expect to find sigmoidal dose-response curves to the measured side effects, and will consider the optimal dose of beclomethasone to be the lowest one that approximates the lower inflection point for multiple readouts. If the inventors do unexpectedly find impairment of the protective effect even at the lowest dose, 0.25 mg, they will use lower doses if there is evidence of reduction in systemic effects at 0.25 mg to attempt to find a dose that blunts systemic side effects without lowering efficacy.

For the acute, dose-escalation toxicity study, if dose-limiting toxicity is detected at twice the therapeutic dose for Francisella species, even after the addition of an inhaled steroid, an analysis will be undertaken to see whether the therapeutic aerosol can be further fractionated to resolve efficacious and toxic components.

For the lung mechanics studies, the inventors do not expect to find substantial changes in static compliance because of the lack of apparent respiratory distress in treated mice in the past, and the lack of histopathologic changes at the alveolar level. If changes in static compliance are nonetheless observed, the inventors will measure lung accumulation of Evans blue dye at the same time points to quantify alveolar capillary permeability changes. If substantial changes in Evans blue dye accumulation are measured, then alveolar capillary permeability changes will be assessed in dogs by Evans blue dye experiments in addition to histopathologic assessment. The inventors consider it more likely that they will observe changes in dynamic compliance, indicating reduced airway caliber. This may occur without intravenous methacholine challenge, suggesting airway wall thickening from inflammation, consistent with what the inventors observe histopathologically. As noted above, the inventors expect this to be modest because of the lack of observed distress in mice treated with aerosolized NTHi lysate. There may also be airway hyperresponsiveness indicated by heightened sensitivity to methacholine, though the inventors do not expect this because of the lack of Th2 inflammation in response to aerosolized NTHi lysate. If there is an increase in either baseline or methacholine-induced dynamic lung compliance, the inventors will test the ability of inhaled steroids or albuterol to attenuate the response.

### Example 11

### Safety Assessment of Aerosolized NTHi Lysate in Dogs

Toxicity testing of aerosolized NTHi lysate in a non-rodent species will be required by the FDA prior to commencing human trials, and dogs are most commonly used for this purpose.

Aerosolized NTHi lysate will be prepared at GMP grade. Efficacy of individual batches will be tested in the laboratory as the ability to confer full protection against challenge with 1×10¹¹ pneumococci four hour after treatment with aerosolized NTHi lysate. Four control and four treated mice will be assessed.

Toxicity testing of aerosolized NTHi lysate in dogs will be conducted. For these studies, aerosolized NTHi lysate will be given through a tight-fitting face mask that will deliver a higher fraction of the aerosol to the lungs than the atmospheric delivery system used in mice. The system to be used in dogs more closely approximates the system the inventors will use in human subjects, which is a simple handle-held nebulizer, such as the AeroMist used in mouse studies, connected to a mouthpiece. The inventors initial study will be a simple dose-finding study using one dog each at four doses of aerosolized NTHi lysate. The studies will use 0.5 mg/ml, where the inventors anticipate no significant toxicity based on preliminary studies in mice; 2.5 mg/ml, which is the therapeutic dose in mice; 10 mg/ml, which is four times the therapeutic does; and 25 mg/ml, which is the maximal concentration the inventors expect to be able to deliver by aerosol, and is ten times the therapeutic dose. Dogs will undergo CT scan of the chest at 24 hours after exposure to aerosolized NTHi lysate, which is the time of maximal inflammation and protection in mice, to look for radiographic evidence of lung injury. Afterwards, they will undergo wedged bronchoalveolar lavage (BAL) for cell counts and proteomic analysis. Dogs will be sacrificed after one week, when the inflammation and protection have mostly resolved in mice. One lung will be harvested for histopathology, and the other for BAL fluid analysis of cells and proteins. Blood will be sampled at 2, 8, 24, 48, and 72 hours, and at the time of sacrifice for routine chemistries and cell counts, as well as for biochemical markers of systemic inflammation such as hepatic acute phase reactants and cytokines. Tissue samples from all organs will be fixed and embedded for histopathologic analysis required by the FDA, and frozen samples for molecular analyses as requested. Further study will include a more substantial toxicity study designed after pre-IND discussions with the FDA. The inventors anticipate using 4 dogs at each of 4 doses ranging from “no observed adverse effect level” based upon mouse studies and the pilot dog study, to the therapeutic dose level, to twice the therapeutic dose level, to the maximal deliverable dose level. Two dogs of each sex will be used at each dose.

Serious pulmonary or systemic toxicity is not expected at the therapeutic dose of 2.5 mg/ml or less, based upon the mouse studies. However, if the inventors do find toxicity at this level, possibly related to more efficient delivery of the aerosol to the lower respiratory tract using the face mask in dogs compared to the atmospheric exposure in mice, additional efficacy studies may be necessary in dogs to see if the efficacious dose is lower than in mice. It is quite possible that pulmonary toxicity will occur at the highest dose levels, due to alveolar inflammation not seen at lower dose levels, and it will be important to identify the threshold for such toxicity. For purposes of comparing doses in dogs and mice, doses can be considered roughly comparable if the concentration of drug and time of delivery are held constant because the size of the lungs (and hence the volume of the inspired dose) between species scales with total body size. However, the inventors will use the level of BAL neutrophils relative to macrophages at 24 hours as a measure of biologic response, along with the relative rise in calgranulin and lysozyme measured by HPLC (FIG. 7) or ELISA. While the inventors do not know whether calgranulin and lysozyme specifically play major roles in protection conferred by aerosolized NTHi lysate, and know that neutrophils do not, all of these serve as measures of the stimulation of inflammatory pathways in the epithelium associated with the protective response.

### Example 12

### Assessment of the Efficacy of Aerosolized NTHi Lysate in a Model of Inhalational Tularemia

It is not possible to perform efficacy studies in humans with highly pathogenic organisms such as Francisella tularensis. To give a high level of confidence that aerosolized NTHi lysate will afford protection against a bioweapon or emergent infection epidemic, an efficacy study in a non-human primate is advisable.

This study will be carried out with a non-human primate inhalational tularemia model. Aerosolized NTHi lysate will be given through a tight-fitting face mask. The inventors will discuss the study design with the FDA, but anticipate using four primates in the control group and four in the group treated with aerosolized NTHi lysate, all of whom will be exposed to Francisella. It is expect that the control animals will die, and that they will be humanely sacrificed when they begin to show serious toxicity. It is also anticipated that the animals treated with aerosolized NTHi lysate will survive, and if so, they will be sacrificed after two weeks because of the risk of harboring Francisella organisms in their fur or elsewhere. One lung will be harvested for histopathology, and the other for BAL fluid analysis of cells and proteins. Blood will be sampled at 2, 8, 24, 48, and 72 hours, and at the time of sacrifice for routine chemistries and cell counts, as well as for biochemical markers of systemic inflammation such as hepatic acute phase reactants and cytokines. Tissue samples from all organs will be fixed and embedded for histopathologic analysis as required by the FDA, and frozen samples for molecular analyses that may be requested.

As above, it is expected that the group of animals exposed to aerosolized NTHi lysate to survive, but to require sacrifice after two weeks. This will provide an opportunity to acquire additional toxicity at the time of sacrifice, when the effects of aerosolized NTHi lysate can be expected to fully resolve according to studies in mice. If the animals are not fully protected by aerosolized NTHi lysate at the anticipated therapeutic dose, which the inventors consider unlikely, higher doses can be attempted depending on the results of the dog toxicity study.

### Example 13

### Assessment of Safety in Human Subjects

### Phase I Study

Assessment in human subjects is required by the FDA prior to commercial development. Such studies will be designed in consultation with the FDA. An exemplary study is outlined below.

The inventors anticipate testing four subjects at each of five doses, ranging from the “no observed adverse effect level” based upon mouse and dog studies, to a dose midway between this and the therapeutic dose established by mouse studies, the therapeutic dose, twice the therapeutic dose, and three times the therapeutic dose. Subjects will be administered the St. George's respiratory questionnaire in addition to a general symptoms questionnaire at baseline, 1 hr., 4 hr., 8 hr., 24 hr., 48 hr., 72 hr., 1 week, and two weeks. Blood will be drawn at all the same intervals for routine chemistry and cell counts, as well as for biochemical markers of systemic inflammation such as hepatic acute phase reactants, and vital signs including pulse oximetry will be recorded. Subjects will be observed for the first 8 hr. onsite, then return for subsequent testing. They will undergo full pulmonary function testing at baseline and 1 week, and spirometry at 1 hr, 4 hr, 8 hr, 24 hr, 48 hr, 72 hr, 1 week, and two weeks. A baseline chest radiographs will be obtained, and if any dyspnea develops, the radiograph will be repeated. A CT scan of the chest will also be obtained if there is evidence of alveolar infiltrates on chest radiograph, or a fall in oxygen saturation >4%, or a fall in lung volumes and diffusing capacity. Subjects will also undergo testing of cognitive function at baseline, 4 hr, 24 hr, and 72 hr to determine whether any systemic inflammation that might be present could affect battlefield performance.

The inventors expect to find evidence of mild systemic inflammation at the doses proposed, manifesting as modest elevations in plasma acute phase reactants and cytokines, and possibly with mild symptoms of fever and lassitude, but no serious toxicity. If systemic inflammation is mild, the inventors do not expect serious impairment of cognitive function. If desired by Department of Defense, cardiopulmonary exercise testing could be undertaken at 4 hr, 24 hr, and 72 hr to determine whether there is any impairment that could affect battlefield performance. If bronchospasm is observed, albuterol and inhaled steroid will be administered, and lung function rechecked. If potentially serious toxicity is observed at any dose level, the FDA will be conferred prior to proceeding to the next dose level.

### Example 14

### Aerosolization of Lysates

When the inventors recognized the powerful protective effect of the aerosolized NTHi lysate against microbial infection, together with its minimal toxicity after short-term use, formulation was prepared as a practical therapeutic. Several fractionation schemes were tried for removal of particulates, and to yield a preparation that could be lyophilized and reconstituted while retaining activity. The aerosolized NTHi lysate preparation generates an odorless, white opalescent liquid that can be lyophilized to a white powder. After reconstitution with water or saline solution, it can be readily resuspended and aerosolized. It can also be reconstituted with prepackaged albuterol and/or steroid ampules if combination therapy is found to be advantageous. It can be aerosolized with commercial nebulizers widely used for bronchodilator therapy, though somewhat more expensive nebulizers such as the AeroMist used in animal studies can generate aerosol droplets of more precise size to limit alveolar exposure. Multiple subjects can be treated consecutively with a single nebulizer simply by changing the mouthpiece. Nebulizers can be powered by compressed gas (oxygen or air) delivered through regulators at 5-10 liters/min, which are found in most patient care areas and inpatient rooms in hospitals. Alternatively, nebulizers can be powered by motorized gas compressors used by many asthma and COPD patients at home, and could be used by the military in the field.

### Example 15

### Confirmation of Protection to Other Organisms

The methods of the invention are effective against a variety of organisms, such as, but not limited to Aspergillus fumigatus, Pseudomonas aeruginosa, Methicillin-resistant Staphylcoccus aureus, Bacillus anthracis, Yersinia pestis, Francisella tularensis, and influenza A. Studies confirming such have been completed using the following general materials and methods.

Animals and Reagents. General reagents are obtained from Sigma Chemical (St Louis, Mo.). Wild-type BALB/c, C57BL/6, and Swiss-Webster mice can be purchased from Harlan (Indianapolis, Ind.).

Organisms. NTHi, A. fumigatus, P. aeruginosa, Methicillin-resistant S. aureus can be obtained from public sources such as American Type Tissue Culture collection and other public depositories or from the United States Government. Pathogen inocula were targeted to induce 75-80% mortality by 48 hours post exposure.

Pseudomonas aeruginosa culture. Bacteria (1×10⁸ CFU/ml stock) were incubated in LB-Medium at 37° C. in 5% CO₂, then diluted in 1 liter of fresh broth and grown in shaker at 37° C. for 6-7 hr to OD₆₀₀ of 0.3, yielding ˜3×10¹⁰ CFU. The suspension was centrifuged, washed with PBS, then resuspended in PBS, and the bacterial concentration was determined by serial dilutions on Tryptic Soy agar plates (Becton Dickinson, Franklin Lakes, N.J.).

Aspergillus fumigatus culture. Fungus was plated on yeast extract medium (YAG) agar plates (Sigma), incubated at 37° C. with 5% CO₂. Plates were harvested by gentle scraping under PBS containing 0.1% Tween 20 and the suspension was filtered, and centrifuged. The supernatant was discarded, the pellet washed with PBS, centrifuged and finally resuspended pellet in PBS. Conidia counts were determined by standard hemacytometer.

P. aeruginosa and A. fumigatus infection model. Mice were infected with P. aeruginosa or A. fumigatus by inhalation. The mice were placed in a sealed nebulization chamber (except for the efflux limb of the nebulizer circuit). An AeroMist CA-209 compressed gas nebulizer (CIS-US, Inc., Bedford, Mass.) aerosolized pathogen suspensions, driven by 10 L/min of room air and supplemented with 5% CO₂ to promote maximal ventilation and homogeneous exposure throughout the lungs 10 ml of the culture suspensions were delivered over 60 minutes.

MRSA culture. S. aureus was grown at 37° C. in CCY medium (3% yeast extract, 2% Bacto-Casamino acids, 2.3% sodium pyruvate, 0.63% Na₂HPO₄ and 0.041% KH₂PO₄ [pH 6.7]). S. aureus were grown at 37° C. to exponential phase OD₆₀₀=1 (˜2 hrs), harvested by centrifugation, washed and resuspended in sterile PBS and diluted accordingly. Anesthetized BALB/c mice (Harlan, Indianapolis, Ind.) were intranasally exposed to S. aureus cells. Criteria for determining morbitity/sickness in mice included hunched posture, decreased activity, ruffled fur and labored breathing. Weight statistical analysis was performed using Student's T-test.

Preparation of B. anthracis spores. Spores were prepared by inoculating B. anthracis in sporulation medium consisting of 16 g Difco Nutrient Broth, 0.5 g MgSO₄.7H₂O, 2.0 g KCl, and 16.7 g MOPS per liter. Before inoculation, the following supplements were added to the medium after filter sterilization using 0.22-μm syringe filters: 0.1% glucose, 1 mM Ca(NO₃)₂, 0.1 mM MnSO₄, and 1 μM FeSO₄. Cultures were grown at 37° C. with gentle shaking (80-90 rpm) for 24 h, after which 100 ml of sterile distilled water was added to dilute the medium and promote sporulation. After 10-11 days of continuous shaking, sporulation was confirmed at >99% via the malachite green spore stain, and the spores were centrifuged in a sealed-carrier centrifuge (Jouan Inc., Winchester, Va.). Spore pellets were then washed four times in sterile phosphate-buffered saline (PBS) and resuspended in the same buffer. B. anthracis cultures and spores were prepared and stored as required by regulations and laws of the United States.

Yersinia pestis culture. To prepare Yersinia pestis culture, bacteria glycerol stock was streaked onto a sheep blood agar plate and incubated at 28° C. for 48 hrs. Colonies were scraped off of the plate using a sterile loop and a suspension was made using heart infusion broth (HIB). A diluted suspension was added to HIB containing 0.2% xylose and allowed to incubate for 24 hrs at 30° C. while shaking at 100 rpm. Following incubation, the suspension was centrifuged at 5000 rpm for 10 minutes, washed twice in 10 ml of HIB and adjusted to an optical density of 10.0 at 620 nm (resuspension of pellet in 10 ml HIB yielding approximately 10¹⁰ CFU/ml). The bacterial pellet was washed in water and 10 μl was saved for serial dilutions and plating. Additional dilutions were made in water to reach the LD₅₀ appropriate for the experimental protocol.

Francisella tularensis culture. Bacteria were grown for 2 days on BHI agar plates enriched with IsoVitaleX. Plate-derived bacteria were then grown in modified Muller Hinton broth (MHB; Difco Laboratories) enriched with IsoVitaleX. The bacteria were grown for 12-15 h, at which time, bacteria consistently reached 2×10⁹-3×10⁹ CFU/ml. The bacteria from cultures at this growth phase in each experiment and the actual concentration of bacteria was verified by a Petroff-Hausser chamber, and plate counts after growing aliquots on BHI plates. Typically, mice were challenged with 5 LD₅₀ doses of Francisella.

Challenge of mice with B. anthracis Ames spores. To evaluate the protective efficacy of NTHi lysate in vivo, 8-week-old (25 to 30-g) female Swiss-Webster mice were challenged (Taconic, Germantown, N.Y.) intranasally with B. anthracis spores.

Mice were anesthetized. Anesthetized animals were suspended vertically, using the upper incisors, as described by Corner et al. The spore suspension was instilled onto the anterior opening of each naris.

NTHi lysate. Was prepared and administered as described above.

Immunosuppression. To achieve immunosuppression, cyclophosphamide (Sigma-Aldrich Inc., St Louis, Mo.) was dissolved in sterile saline to a concentration of 15 mg/ml and administered by intraperitoneal before A. fumigatus aerosolization. Cortisone acetate (Sigma-Aldrich Inc.) was suspended in sterile saline containing 0.1% Tween 20 to a concentration of 60 mg/ml and administered by subcutaneous injections (300 μg/mg) also on days −4 and −1 prior to infection.

Galactomannan assay. The single-incubation sandwich ELISA procedure for Aspergillus galactomannan detection was adapted from the commercially-available Platelia EIA kit (BioRad Laboratories, Redmond Wash.).

Quantitative PCR. Pulmonary fungal burden was determined by real-time qPCR as previously described.

Histology. Following immunosuppression with cyclophosphamide and cortisol, Swiss-Webster mice were challenged with A. fumigatus with or without NTHi lysate pretreatment, as described above. 24 h post-challenge, the mice were anesthetized, exsanguinated, and their pulmonary circulation was perfused with PBS. The lungs were fixed in situ with 4% paraformaldehyde at a pressure of 10 cm H₂O, removed from the thorax, and fixed overnight at 4° C. The fixed lungs were parrafin-embedded, cut into 5 μm serial sections, and applied to Superfrost Plus microscope slides. The samples were then submitted to histological inspection following staining with Gomori methenamine silver (GMS) or hematoxylin-eosin.

Gene Expression Analysis. To evaluate host responses to the NTHi lysate, whole genome oligonucleotide gene expression microarray analysis was performed. At designated time points after the treatment, C57BL/6 mice were anesthetized, their pulmonary vasculature was exsanguinated, and they were submitted to repeated BAL to reduce the leukocyte burden of the airspaces. The lungs were then excised, homogenized, and total RNA was isolated using the RNeasy system (Qiagen, Valencia, Calif.). cRNA was synthesized, then amplified, from equal masses of total RNA extracted from the lungs of infected/sham challenged mice using the Ilumina TotalPrep RNA amplification kit (Ambion, Austin, Tex.). Amplified cRNA was then hybridized and labeled on Sentrix Mouse-6 Expression BeadChips (Illumina, Inc., San Diego, Calif.). All microarrays were scanned on a BeadStation 500 (Illumina). Analysis of the microarray output was performed using an ANOVA-based schema to identify infection-induced changes written in R (Free Software Foundation, Boston, Mass.), utilizing the lumi library developed by Dr Simon Lin, Northwestern University. All primary expression microarray data are available online at the NCBI Gene Expression Omnibus (ncbi.nlm.nih.gov/geo/) in accordance with MIAME (minimal information about a microarray experiment) standards. Pathway analyses were performed by multiple strategies. Primary gene ontology for function, cellular location and general transcript mechanism class were performed using the NIAID Database for Annotation, Visualization and Integrated Discovery (DAVID). Using GenBank accession numbers, DEGs were subsequently mapped to signaling pathways using Ingenuity Pathways Analysis 5.0 (Ingenuity Systems, Redwood City, Calif.) and KEGG (GenomeNet, Kyoto, Japan) software. Finally, pathway predictions from the three prior systems were combined with expert predictions into R code developed by Dr Paul Gold to identify additional involved pathways.

Statistical Analysis. Survival experiments were treated as categorized time-to-event data. To compare the treatment effect on survival time, the Mantel-Cox test was used. Estimates of common relative risk (odds ratio) between groups and its 95% confidence intervals prepared. The cumulative death rates (defined as the cumulative number of death at time t divided by the total number) were plotted. Survival plots were created in SPLUS. All statistical analyses were performed in SAS® Version 9.1 (SAS Institute, Cary, N.C.), and SigmaPlot® 10 (Systat, San Jose, Calif.).

### REFERENCES

The following references, to the extent that they provide exemplary procedural or other details supplementary to those set forth herein, are specifically incorporated herein by reference.
U.S. Pat. No. 4,668,218
U.S. Pat. No. 5,458,135
U.S. Pat. No. 6,488,953
U.S. Pat. No. 6,737,045
U.S. Pat. No. 6,794,357
U.S. Pat. No. 6,797,258
Dennis et al. JAMA 285:2763-2773, 2001.
EP 0237507
Martin and Frevert, Proc Am Thorac Soc 2:403-411, 2005.
PCT Appln. WO 94/06498
PCT Appln. WO 94/08552
PCT Appln. WO 94/16970
PCT Appln. WO 97/25086
PCT Appln. WO 98/35888
Physicians' Desk Reference

1. A method of attenuating respiratory infection by a first microbe in a human subject who has or is at risk for developing such an infection, the method comprising administering a microbial lysate, wherein said lysate is prepared from a second microbe, to the subject by aerosol inhalation in an amount sufficient to induce innate immunity in the subject to said first microbe and thereby attenuate the respiratory infection.

2. The method of claim 1, wherein the subject has been exposed to a pathogenic microbe.

3. The method of claim 1, wherein the lysate is administered to the subject before the subject is exposure to the first microbe.

4. The method of claim 1, wherein the first microbe is a virus, a bacteria, or a fungus.

5. The method of claim 4, wherein the microbe is a virus.

6. The method of claim 5, wherein the virus is Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae.

7. The method of claim 6, wherein the virus is Parainfluenza, Influenza, H5N1, Marburg, Ebola, Severe acute respiratory syndrome coronavirus, Yellow fever, Human respiratory syncytial, Hantavirus, or Vaccinia virus.

8. The method of claim 4, wherein the microbe is a bacteria.

9. The method of claim 8, wherein the bacteria is an intracellular, a gram positive or a gram negative bacteria.

10. The method of claim 9, wherein the bacteria is a Staphylococcus, a Bacillus, a Francisella, or a Yersinia bacteria.

11. The method of claim 10, wherein the bacteria is Bacillus anthracis, Yersinia pestis, Francisella tularensis, or Staphylococcus aureas.

12. The method of claim 11, wherein the bacteria is Bacillus anthracis.

13. The method of claim 11, wherein the bacteria is Staphylococcus aureas.

14. The method of claim 4, wherein the first microbe is a fungus.

15. The method of claim 14, wherein the fungus is a Aspergillus, Candida, Cryptococcus, Histoplasma, Coccidioides, Blastomyces, Zygometes, or Pneumocystis.

16. The method of claim 1, wherein the microbial lysate is administered to the subject at least 2 times.

17. The method of claim 1, wherein the subject is immunocompromised.

18. The method of claim 17, wherein the subject is infected with an immunodeficiency virus.

19. The method of claim 1, wherein the second microbe is a bacteria.

20. The method of claim 19, wherein the bacteria is a non-typeable Haemophilus influenzae (NTHi), Acetobacter aceti, Bacillus cereus, B. licheniformis, B. megaterium, B. pumilus, B. subtilis, Erwinia dissolvens, Lactobacillus acidophilus, L. bulgaricus, L. casei, L. delbrückii, L. helveticus, L. lactis, Leuconostoc, L. mesenteroides, Pediococcus, Propionibacterium acidipropionici, P. freundenreich ii, P. jensenii, P. shermanii, P. technicum, P. thoenii, Streptococcus cremoris, S. diacetilactis, S. faecalis, S. lactis, or S. thermophilus, or Escherichia coli.

21. The method of claim 20, wherein the bacteria is a non-typeable Haemophilus influenzae (NTHi).

22. The method of claim 1, wherein the second microbe is a virus.

23. (canceled)

24. A pharmaceutically acceptable composition comprising a lysate of microbe, an anti-inflammatory agent and one or more pharmaceutical excipients, wherein said composition is sterile and essentially free of pathogenic microbes.

25.-27. (canceled)

28. A pharmaceutically acceptable aerosol composition prepared by a process comprising the steps of:
(a) obtaining a composition of essentially non-pathogenic microbe;
(b) treating the composition to kill microbes therein;
(c) lysing the microbes to prepare a lysate; and
(d) aerosolizing the lysate to prepare the aerosol composition;
wherein the aerosol composition is sterile and essentially free of pathogenic microbes.

29.-35. (canceled)

36-40. (canceled)

41. A method of preparing a pharmaceutically acceptable aerosol composition in accordance with claim 24, comprising the steps of:
(a) obtaining a composition of essentially non-pathogenic microbe;
(b) treating the composition to kill microbes therein;
(c) lysing the microbes to prepare a lysate; and
(d) aerosolizing the lysate to prepare the aerosol composition;
wherein the aerosol composition is sterile and essentially free of pathogenic microbes.

42.-44. (canceled)
